(19) United States
(12) Patent Application Publication
(10) Pub. No.: US 2025/0051862 A1
(43) Pub. Date: Feb. 13, 2025

(54) METHODS AND COMPOSITIONS RELATED TO ENGINEERED BIOSENSORS

(71) Applicant: BOARD OF REGENTS, THE UNIVERSITY OF TEXAS SYSTEM, Austin, TX (US)

(72) Inventors: Andrew ELLINGTON, Austin, TX (US); Simon D'OELSNITZ, Austin, TX (US); Shaunak KAR, Austin, TX (US); Ross THYER, Houston, TX (US)

(21) Appl. No.: 18/566,011

(22) PCT Filed: Jun. 2, 2022
(86) PCT No.: PCT/US2022/031957

§ 371 Date: Nov. 30, 2023

Related U.S. Application Data
(60) Provisional application No. 63/196,001, filed on Jun. 2, 2021.

(51) Int. Cl. C12Q 1/6897; C12N 15/70

(52) U.S. Cl. CPC C12Q 1/6897; C12N 15/70; C12N 2503/00; G01N 2333/415

(57) ABSTRACT

Disclosed herein are substrate-promiscuous regulators which have been engineered to function as highly efficient biosensors. These engineered biosensors are significantly more specific to the target ligand than their naturally occurring counterparts, and are able to generate a detectable output signal upon exposure to the input signal (target ligand). Also disclosed of methods of making engineered biosensors based on a naturally occurring substrate-promiscuous regulator. Also disclosed are methods of using these biosensors to make a product, such as cell-based bioengineering platforms. Lastly, disclosed are kits, nucleic acids, and proteins related to the biosensors disclosed herein.

### CROSS-REFERENCE TO RELATED APPLICATIONS

This application is a U.S. National Stage application filed under 35 U.S.C. § 371 of PCT/US2022/031957 filed Jun. 2, 2022, which claims the benefit of U.S. Provisional Application No. 63/196,001, filed Jun. 2, 2021, each of which is incorporated herein by reference in their entirety.

### GOVERNMENT SUPPORT CLAUSE

This invention was made with government support under Grant no. FA9550-14-1-0089 awarded by the Air Force Office of Scientific Research, and Grant no. HR0011-19-2-0019 awarded by the Defense Advanced Research Projects Agency (DARPA). The government has certain rights in the invention.

### REFERENCE TO AN ELECTRONIC SEQUENCE LISTING

The content of the electronic sequence listing submitted on Jul. 1, 2024, as a text file named “10046-413US1-PAPER” created on Jun. 26, 2024, and having a size of 284 KB, is hereby incorporated by reference in its entirety pursuant to 37 CFR 1.52 (e) (5).

### BACKGROUND

Microbes have been extensively engineered for commercial-scale production of therapeutic plant metabolites, yielding many benefits over traditional plant cultivation methods, such as reduced water and land use, faster and more reliable production cycles, and higher purity of target metabolites. Microbial fermentation is currently used for the production of artemisinic acid, the immediate precursor to the antimalarial drug artemisinin, and in development for commercial production of cannabinoids, opiates, and tropane alkaloids [1-5]. However, scaling production typically requires several years and hundreds of person-years to complete [5], and is largely bottlenecked by a reliance on low-throughput analytical methods for assessing strain and pathway performance [6]. Prokaryotic transcriptional regulators have been repurposed as biosensors to address this limitation for certain metabolites by enabling high-throughput screens within living cells [7]. However, for virtually all therapeutic plant metabolites there exists no corresponding biosensor, since most sensors are largely restricted to compounds hardwired into microbial metabolism. Although genetic biosensors have been evolved to recognize alternative ligands, these are typically modest changes compared to their cognate ligand [8]. Therefore, a new approach to sensor engineering is needed to realize high-throughput engineering of therapeutic plant metabolite pathways.

A protein's substrate promiscuity is thought to strongly correlate with its evolvability [9]. Therefore, the evolutionary specialization of hyper-promiscuous biosensors may be a powerful generalizable strategy to generate custom sensors for user-defined analytes. This approach has already been applied to rapidly evolve enzymes for unnatural compounds. Classic examples of this include the evolutionary work with the cytochrome protein P450-BM3, where just a single point mutation increased the enzyme's non-natural cyclopropanation activity more than 60-fold [10], and the evolution of the serum paraoxonase 1 for hydrolysis of synthetic organophosphates, improving the catalytic activity by ˜105 following several rounds of directed evolution [11]. Despite the pressing need to expand the chemical scope of genetic biosensors, this approach has not yet been thoroughly explored for biosensor engineering.

The biosensor equivalent of hyper-promiscuous and highly evolvable enzymes are prokaryotic multidrug resistance regulators, typically studied as mediators of broad-spectrum antibiotic resistance. These regulators characteristically have large substrate binding pockets which often recognize structurally-diverse lipophilic molecules via non-specific interactions [12]. Early studies also suggest that they are highly evolvable. Notably, a single point mutation enabled one of these regulators to adopt a substantial affinity for a non-cognate ligand [13]. What is needed in the art are engineered substrate-promiscuous regulators that can be used in the production of target molecules.

### SUMMARY

Disclosed herein is a biosensor comprising an engineered substrate-promiscuous regulator, wherein said substrate-promiscuous regulator has been engineered to interact more efficiently with an input signal than does the naturally occurring substrate promiscuous regulator; and further wherein the biosensor is engineered to provide an output signal, wherein said output signal is generated in response to interaction with the input signal.

Also disclosed is a method of engineering a substrate-promiscuous regulator to function as a biosensor, the method comprising: identifying a naturally occurring substrate-promiscuous regulator; engineering the naturally occurring substrate-promiscuous regulator for increased sensitivity to an input signal when compared to the naturally occurring substrate promiscuous regulator; and introducing into a cell: a nucleic acid encoding the engineered substrate-promiscuous regulator, a transduction system for providing an output signal, wherein said output signal is generated in response to interaction with the input signal; and exposing the cell to the input signal; and detecting an output signal; wherein detection of said output signal indicates a functional biosensor.

Further disclosed is a kit comprising: a biosensor comprising an engineered substrate-promiscuous regulator, wherein said substrate-promiscuous regulator has been engineered to interact more efficiently with an input signal than does the naturally occurring substrate promiscuous regulator; and an output signal; wherein said output signal is generated in response to interaction with the input signal.

### DESCRIPTION OF DRAWINGS

FIG. 1A-D shows screening identifies a Benzylisoquinoline-responsive biosensor. (a) Structures of five BIAs used in the screen. (b) Schematic of the genetic circuit used for screening the responsiveness of candidate sensors to target BIAs. (c) Fluorescence response of six biosensors to all five BIAs. Ligand concentrations used for induction are indicated as follows. Glaucine: 1 mM, noscapine: 100 uM, papaverine: 500 uM, rotundine: 250 uM, tetrahydropapaverine: 1 mM. Fluorescence values are the averages of three biological replicates (d) The global structure (left) and ligand binding pocket (right) of RamR in complex with berberine (PDB: 3VW2). Colored residues were targeted for mutagenesis.

FIG. 2A-D shows the SELIS approach for biosensor evolution (Seamless Enrichment of Ligand-Inducible Sensors). (a) Libraries are generated and transformed into E. coli cells (b) Cells containing the sensor library are cultured in the presence of zeocin. Transcriptional repression by sensor variants prevents the expression of Lambda cl, which enables the expression of Sh Ble and confers zeocin resistance. Cells containing sensor variants that are unable to repress are eliminated from the population. Adding non-target ligands at this stage enables counter-selection for specificity. (c) Binding of the sensor variant to the target ligand relieves repression of GFP expression, producing fluorescence. Cultures are plated on an LB agar plate containing the target ligand and highly fluorescent colonies are cultured overnight. Subsequently, cultures from each picked colony are split and grown either with or without the target ligand. (d) Variants that display high signal/noise ratios are sequenced, subcloned, and re-phenotyped with a wider range of ligand concentrations. The top performing variant is used for the next cycle of evolution.

FIG. 3A-E shows evolution of highly specific BIA sensors from a generalist template. (a) Transfer functions for all four generations of RamR variants with five different BIAs. The maximum ligand concentration was chosen based on the compound's solubility limit in 1% DMSO, 100 μM for Noscapine and 250 μM for all other BIAs. Fluorescent measurements for each condition were an average of three biological replicates. (b) Background fluorescence measurements for all RamR generations. The same promoter was used to express variants from each evolutionary trajectory (see methods) (c) Orthogonality matrix of all evolved sensors. Fold-response is shown for all BIAs for the native RamR protein, the first, second, third, and fourth generations from top to bottom, respectively. 100 μM of the indicated BIA was applied in all conditions. Measurements for each condition were an average of three biological replicates.

FIG. 4A-C shows crystal structures of evolved biosensors bound to cognate benzylisoquinoline alkaloids. (a) Overall structures of the four evolved RamR variants in ribbon diagram. The specific ligand for each variant is shown in stick with the binding site for one of the monomer shown in space-filling model to highlight the binding pockets. (b) Omit Fo-Fc map (contoured at 3.00) shown as a green mesh superimposed on the stick model of papaverine molecule (carbon atoms in yellow, oxygen atoms in red, and nitrogen atom in blue). (c) Superimposed structures of the complexes with the side chains of residues 70, 85, 133, and 134 in stick with color scheme as PAP4 (red), NOS4 (yellow), ROTU4 (green), and GLAU4 (purple). The isoquinoline ring part of all four ligands (yellow stick) are shown as space-filling with isoquinoline shown in stick (color scheme identical as b). The side chain of F155 π-π stacking with the isoquinoline ring is shown in stick and colored gray.

FIG. 5A-D shows unique molecular adaptations confer alkaloid specificity. (a-d) Structure of evolved sensors in complex with their cognate BIAs (shown in stick with carbon atoms colored yellow, oxygen atoms in red and nitrogen in blue). Residues involved in specific interactions with the cognate ligand are displayed in stick and labeled.

FIG. 6 shows benzylisoquinoline pathway map. Arrows represent enzymatic steps and grey circles represent metabolites. Alkaloids focused on in this work are highlighted with a colored border.

FIG. 7A-B shows multidrug resistance regulator design and validation. (a) Promoter design for each regulator. −35 and −10 promoter regions are highlighted with a red and yellow box, respectively. Operator sequences are underlined. All promoters are followed by the RiboJ riboregulator, a medium strength RBS, the sfGFP gene, and a strong terminator. (b) Validation of promoter activity and regulator repression in E. coli. Cells were co-transformed with the regulator's promoter and either an empty vector (− Sensor) or a vector expressing the cognate regulator (+ Sensor) and promoter activity was monitored via fluorescence.

FIG. 8A-B shows negative selections with the pSelis plasmid. Cells co-transformed with both pReg expressing a library of RamR variants and the pSelis plasmid were grown for 20 hours in the presence of variable amounts of zeocin and fluorescence (a) and cell density (b) were monitored. The “1×” concentration represents 100 μg/mL of zeocin. Assays were performed in biological triplicate.

FIG. 9 shows visual representation of libraries used throughout evolution. (top left) A magnified structure of RamR bound to berberine (PDB: 3VW2) displays residues targeted for mutagenesis. These residues were chosen based on their proximity to berberine. (top middle) global structure of RamR. (top right) The mapping of library color code to the corresponding residues targeted for combinatorial site saturation mutagenesis. (bottom) Libraries used and fixed during evolution. Colored vertical lines represent libraries used to introduce diversity prior to selection. Colored horizontal lines represent library positions fixed.

FIG. 10A-F performance of all top GLAU variants recovered. (a-d) Dose response functions of top unique GLAU variants. Variants were chosen based on their signal/noise ratio measured during evolution (See FIG. 2c). All variants were subcloned into a fresh pReg backbone prior to characterization with the pGFP plasmid. The “x2” symbol denotes that this amino acid sequence was recovered twice following evolution. The variant genotype highlighted in green was chosen as the template for the following round of evolution. Dose response measurements were performed in biological triplicate. (e,f) Selectivity of generation three and four sensor variants. Cells were induced with 100 uM of all non-target BIAs, separately.

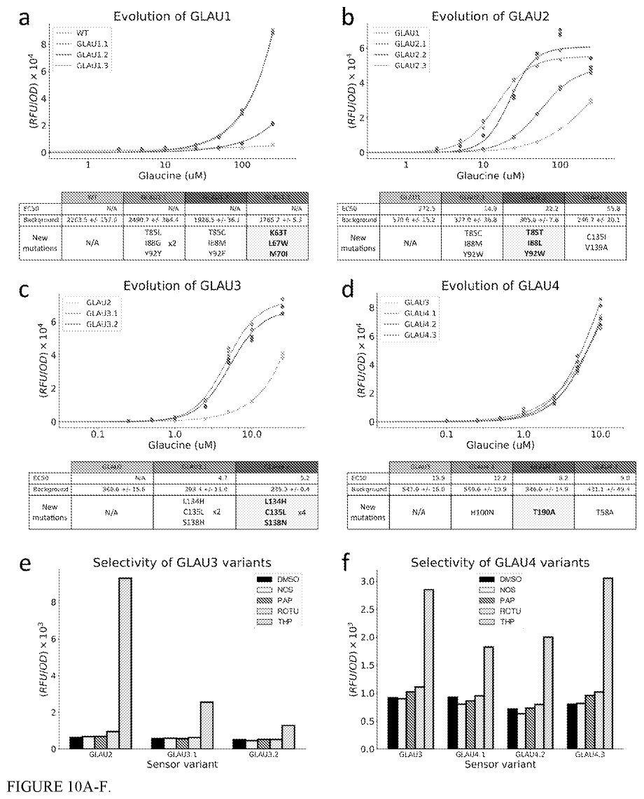

FIG. 11A-F shows performance of all top NOS variants recovered. See FIG. 11A-F legend for details.

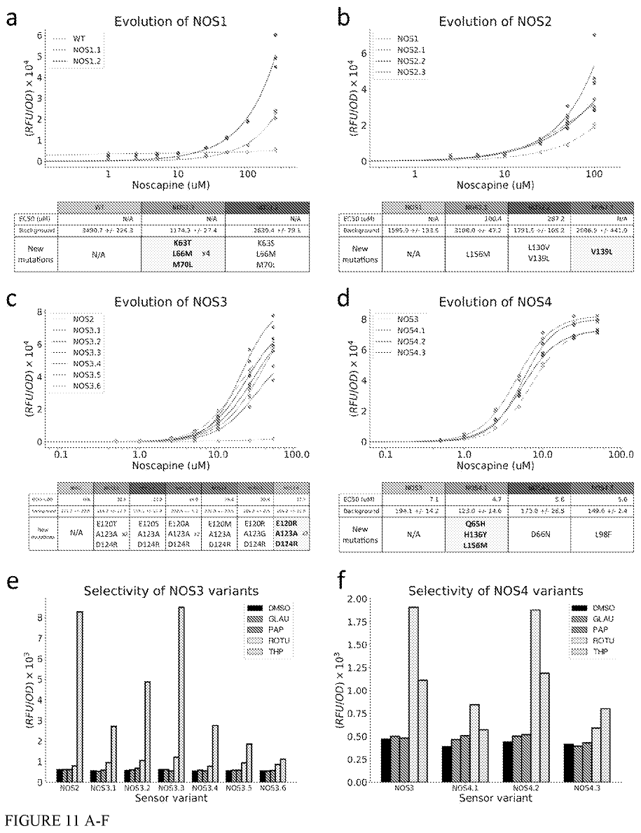

FIG. 12A-F shows performance of all top PAP variants recovered. See FIG. 11A-F legend for details.

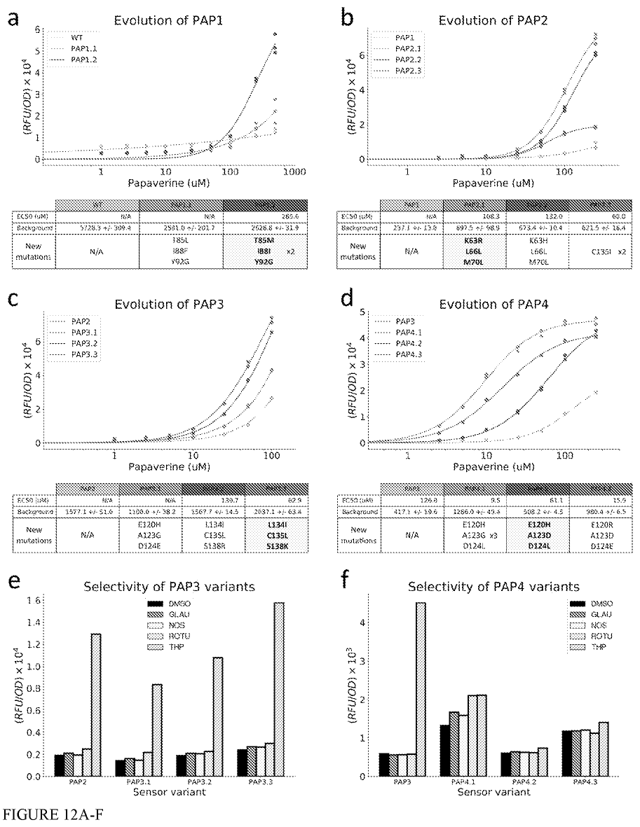

FIG. 13A-F shows performance of all top ROTU variants recovered. See FIG. 11A-F legend for details.

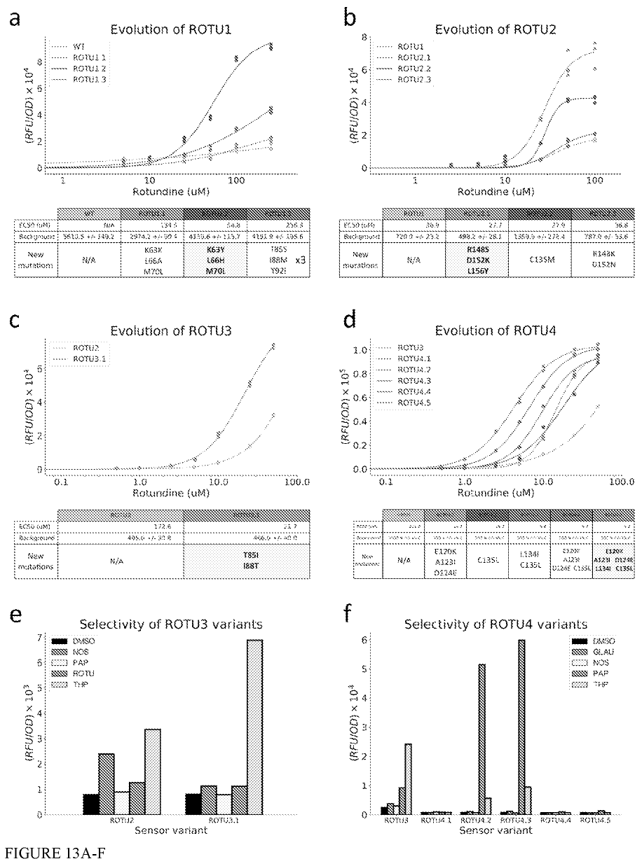

FIG. 14A-F shows performance of all top THP variants recovered. See FIG. 11A-F legend for details.

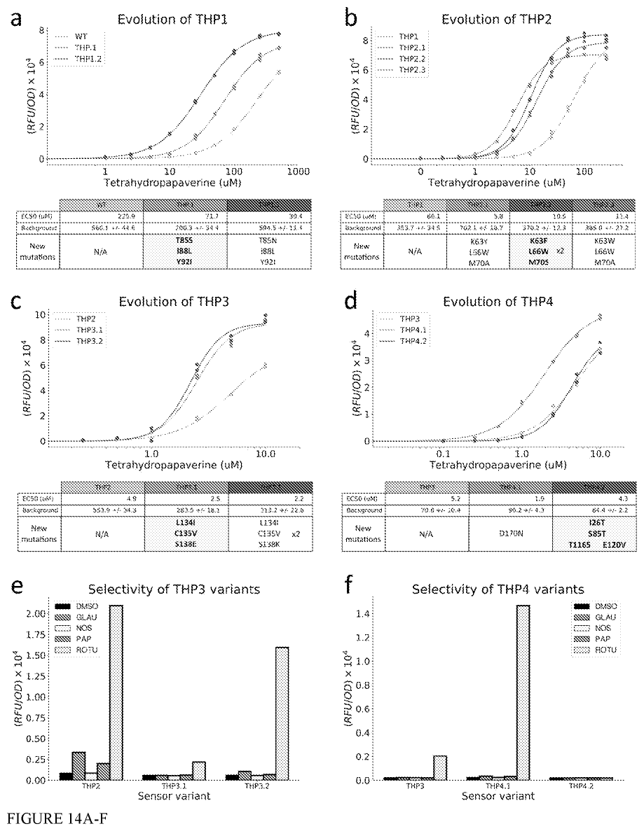

FIG. 15A-E shows orthogonality of all final RamR variants. Fluorescent response of cells expressing pGFP and pReg with WT RamR (a), Gen1 variants (b), Gen2 variants (c), Gen3 variants (d), and Gen4 variants (e) that were induced with 100 uM of each BIA, separately. Measurements were performed in biological triplicate. See “Example 1: Methods-Orthogonality Assays” for the list of promoters used to express each variant.

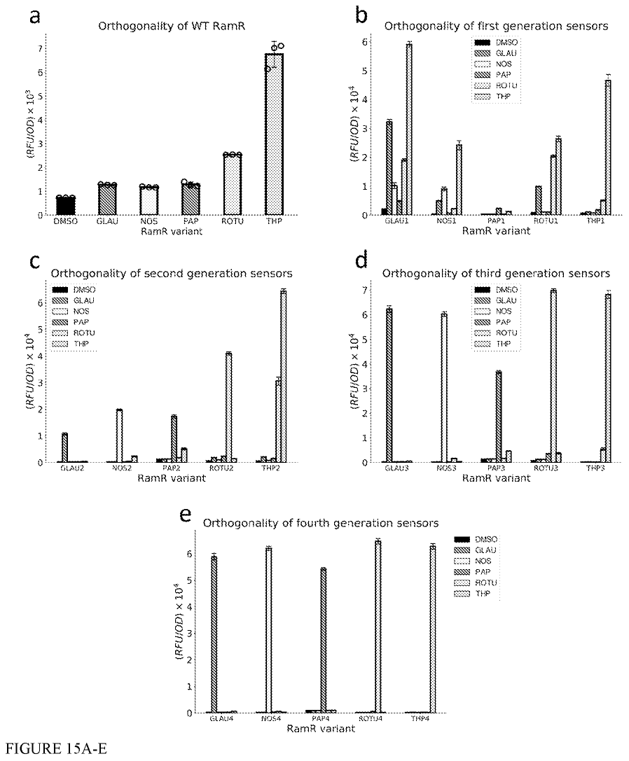

FIG. 16A-C shows A) the chemical synthesis of 4-Omethyl-Norbelladine; B) the response of RamR to amaryllidaceae; and C) amaryllidaceae alkaloid.

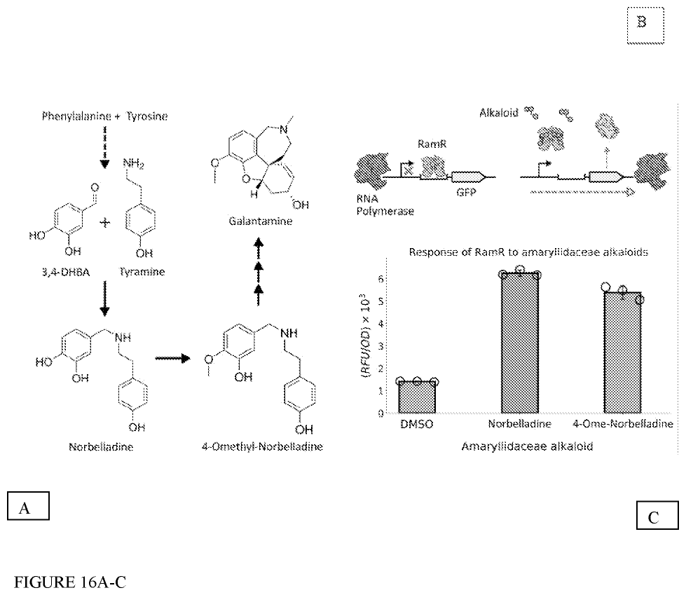

FIG. 17A-B shows A) response to Gen1 4-omethylnorbelladine sensors, and B) selectivity of Gen1 4-Omethylnorbelladine sensors.

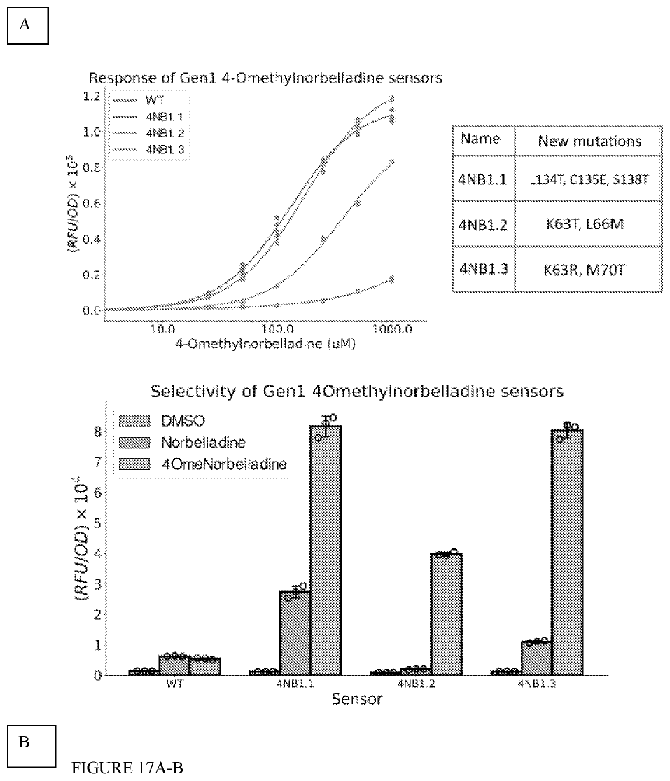

FIG. 18A-B shows A) dose response of Gen2 sensors to 4-Ome-Norbelladine; and B) selectivity of evolved biosensors.

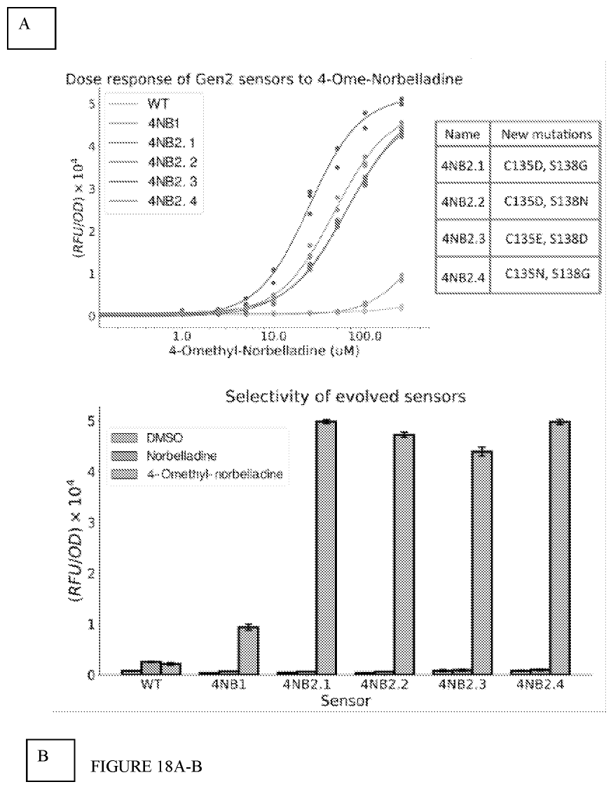

FIG. 19 show that RamR is responsive to numerous alkaloids.

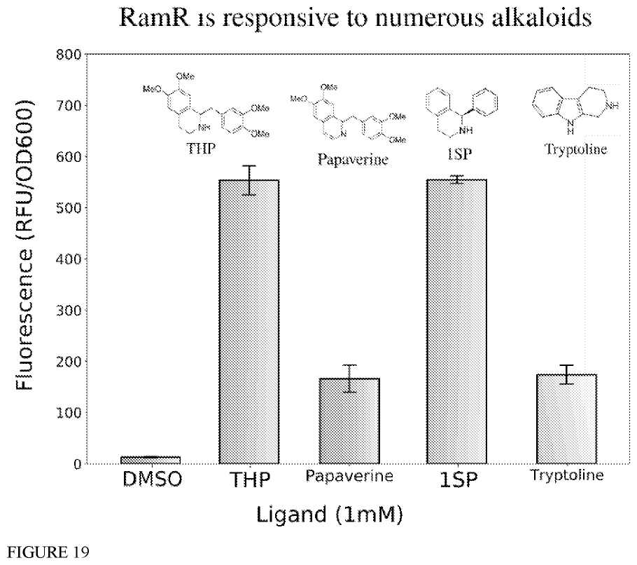

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur.

The disclosed technology relates to “biosensors.” As disclosed herein, a “biosensor” is a molecule or a system of molecules that can be used to bind to a ligand (or target molecule) and provide a detectable response based on binding the ligand. In some cases, “biosensors” may be referred to as “molecular switches.” Biosensors and molecular switches are disclosed in the art. (See, e.g., Ostermeier, Protein Eng. Des. Sel. 2005 August; 18 (8): 359-64; Wright et al., Curr. Opin. Chem. Biol. 2007 June; 11 (3): 342-6; Roberts, Chem. Biol. 2004 November; 11 (11): 1475-6; and U.S. Pat. Nos. 8,771,679; 8,679,753; and 8,338,138; the contents of which are incorporated herein by reference in their entireties). Biosensors and molecular switches have been utilized in recombinant microorganisms. (See, e.g., Rogers et al., Curr. Opin. Biotechnol. 2016 Mar. 18; 42:84-91; and U.S. Published Application Nos. 2010/0242345 and 2013/0059295; the contents of which are incorporated herein by reference in their entireties).

A “substrate-promiscuous regulator” refers to any protein with the ability to bind to and report on the concentration of more than one chemical. For instance, the naturally occurring promiscuous regulators from which the biosensors disclosed herein are derived has been reported to bind to several different unrelated chemicals (Yamasaki, S., Nikaido, E., Nakashima, R. et al. Nat Commun 2013) Another common feature of substrate-promiscuous regulators is that the chemicals they bind are often structurally unrelated, but share some common general feature, such as being hydrophobic.

The systems, components, and methods disclosed herein may be utilized for sensing a ligand or a substrate or a metabolite in a cell or a reaction mixture. The disclosed systems, components, and methods typically include and/or utilize an engineered (non-naturally occurring) biosensor. The biosensors disclosed herein bind the ligand and modulate expression of an output signal, such as a reporter gene, which can be operably linked to a promoter that is engineered to include specific binding sites for the input signal. The difference in expression of the output signal in the presence of the ligand versus expression of the output signal in the absence of the ligand can be correlated to the concentration of the ligand in a reaction mixture.

As used herein, “modulating expression” may include “repressing expression” and/or “inhibiting expression,” and “modulating expression may include “de-repressing expression” and/or “activating expression.” As such, in some embodiments, when the biosensor is not bound to a ligand, the biosensor may repress expression and/or inhibit expression from a promoter that is engineered to include specific binding sites for the DNA-binding protein, and when the biosensor is bound to the ligand the biosensor may de-repress and/or activate expression from the promoter. De-repression and/or activation of the expression of the reporter gene then can be correlated with the presence of the ligand. In other embodiments, when the biosensor is bound to a ligand, the biosensor may repress expression and/or inhibit expression, and when the biosensor is not bound to the ligand the biosensor may de-repress expression and/or activate expression. A decrease in expression of the reporter gene then can be correlated with the presence of the ligand.

The disclosed biosensors, systems, and methods may be utilized and/or performed using any suitable cell. Suitable cells may include prokaryotic cells and eukaryotic cells.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Reference also is made herein to peptides, polypeptides, proteins and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue. For example, a fragment may comprise from 5 to 1000 contiguous nucleotides or contiguous amino acid residues of a reference polynucleotide or reference polypeptide, respectively. In some embodiments, a fragment may comprise at least 5, 10, 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the 5′-terminal region and/or 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides. An insertion or addition may refer to 1, 2, 3, 4, 5, 10, 20, 30, 40, 50, 60, 70, 80, 90, 100, 150, or 200 amino acid residues or nucleotides.

Fusion proteins and fusion polynucleotides also are contemplated herein. A “fusion protein” refers to a protein formed by the fusion of at least one peptide, polypeptide, protein or variant thereof as disclosed herein to at least one molecule of a heterologous peptide, polypeptide, protein or variant thereof. The heterologous protein(s) may be fused at the N-terminus, the C-terminus, or both termini. A fusion protein comprises at least a fragment or variant of the heterologous protein(s) that are fused with one another, preferably by genetic fusion (i.e., the fusion protein is generated by translation of a nucleic acid in which a polynucleotide encoding all or a portion of a first heterologous protein is joined in-frame with a polynucleotide encoding all or a portion of a second heterologous protein). The heterologous protein(s), once part of the fusion protein, may each be referred to herein as a “portion”, “region” or “moiety” of the fusion protein.

A fusion polynucleotide refers to the fusion of the nucleotide sequence of a first polynucleotide to the nucleotide sequence of a second heterologous polynucleotide (e.g., 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein.

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit or more biological activities associated with binding a ligand and/or binding DNA at a specific binding site.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon.

A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein.

“Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 1 3, Cold Spring Harbor Press, Plainview N. Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

### Engineered Biosensors

Disclosed herein is a biosensor comprising an engineered substrate-promiscuous regulator, wherein said substrate-promiscuous regulator has been engineered to interact more efficiently with an input signal than does the naturally occurring substrate promiscuous regulator; and further wherein the biosensor is engineered to provide an output signal, wherein said output signal is generated in response to interaction with the input signal.

Designing genetic biosensors is known in the art (Hossain et al., “Genetic Biosensor Design for Natural Product Biosynthesis in Microorganisms, Trends in Biotechnology 38 (7), p797-810, April 2020, herein incorporated by reference in its entirety for its teaching concerning biosensors). A genetic biosensor is made up of a sensing device and a transduction device, which can be formed by genetic parts. The sensing device serves to detect the existence of an input signal such as a ligand. It contains a TF (transcriptional activator, transcriptional repressor) consisting of a DNA-binding domain (DBD) and a ligand-binding domain (LBD), or an element such as a riboswitch comprising an RNA aptamer. The transduction device translates the input signal into an output signal (e.g., fluorescence, colorimetry, or a genetic trait, such as antibiotic resistance, for example). It contains a reporter gene or pathway genes. The sensing device can be functionally linked to the transduction device through the binding of the input signal to a TF or a riboswitch, for example, activating or repressing transcription or translation of genes of interest. In TF-based biosensors, mediated by DBD and/or LBD, transcriptional activators activate transcription of reporter genes by binding to promoters, and transcriptional repressors repress transcription of actuator genes by dissociating from promoters or binding to a co-repressing ligand in an allosteric manner.

It was discovered that substrate-promiscuous regulators can be used as a starting platform to engineer biosensors that are specific for a certain ligand (referred to alternatively herein as a target). Because these promiscuous regulators can have a high degree of evolvability, they can be engineered with relative ease to be specific for a ligand. In one example, a person of skill in the art can identify a potential substrate-promiscuous regulator that can be engineered for a specific ligand by identifying a substrate promiscuous regulator that shows some degree of affinity for the ligand, then evolving the substrate-promiscuous regulator through mutation to create a biosensor with a much higher degree of specificity for the ligand than the naturally occurring regulator. For example, the engineered substrate-promiscuous regulator can be 2, 3, 4, 5, 6, 7, 8, 9, 10, 20, 30, 40, 50, 60, 70, 80, 90, or 100 times (or more) more efficient at interacting with the ligand than the naturally occurring regulator.

In one example, the substrate-promiscuous regulator disclosed herein can be a genetically engineered multidrug resistance regulator (MDR). Multidrug resistance regulators are known to recognize structurally diverse ligands, however, the extent to which their ligand specificity can adapt has previously remained unexplored. Regulators in this family contain a poly-specific substrate binding pocket that enables them to bind and extrude a diverse array of compounds from the periplasm to the exterior of the cell, including the majority of clinically used antibiotics (Aron et al., Res Microbiol. 2018 September-October; 169 (7-8): 393-400). In order to have utility in microbial engineering for plant metabolites, sensors must be highly specific and sensitive to their target molecule to avoid false positives and report on low-activity pathways, respectively, making multidrug resistance regulators an ideal candidate for engineered biosensors. In a specific example, the substrate-promiscuous regulator can comprise a large hydrophobic binding pocket that contains numerous aromatic residues, such as phenylalanine, tyrosine, and/or tryptophan

Examples of naturally occurring multidrug resistance regulators that can be used as a platform from which to engineer the biosensors of the present invention include, but are not limited to, QacR (WP_001807342.1), TtgR (WP_010952495.1), SmeT (WP_005414519.1), NalD (WP_003092152.1), LmrR (WP_011834386.1), EbrR (WP_003976902), MexR (WP_003114897.1), LadR (WP_003721913.1), VceR (WP_001264144.1), MttR (WP_003693763.1), AcrR (WP_000101737), MepR (WP_000397416.1), SCO4008 (WP_011029378.1), Rv3066 (WP_003416005.1), CgmR (WP_011015249.1), CmeR (WP_002857627.1), Rv0302 (WP_003401571.1), BepR (WP_004687968.1), MexL (WP_003092468.1), TtgT (WP_012052586.1), TtgV (WP_014003968.1), LmrA (WP_003246449.1), TM_1030 (WP_010865247.1) or Bm3R1 (WP_013083972.1), or RamR (WP_000113609.1)

The engineered biosensor can have 90, 91, 92, 93, 94, 95, 96, 97, 98, or 99% identity with a naturally occurring substrate-promiscuous regulator. Viewed another way, the engineered biosensor can vary from a naturally occurring substrate-promiscuous regulator by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. This variation can be in the form of an insertion, deletion, or substitution, or a combination of two or more of these. Given the teachings disclosed herein, one of skill in the art can readily engineer a naturally occurring substrate promiscuous regulator to be highly specific for a desired target molecule (ligand).

The “input signal” is any substance, compound, or composition which one would like to detect. This input signal can be a naturally occurring composition, or it can be a synthetic composition. For example, a naturally occurring composition that can be an input signal in the present invention is a plant alkaloid, such as a benzylisoquinoline alkaloid. Examples of plant alkaloids can be found in Hagel et al (Plant and Cell Physiology, Volume 54, Issue 5, May 2013, Pages 647-672), which is hereby incorporated by reference in its entirety for its teaching concerning benzylisoquinoline alkaloids. In one embodiment, the plant alkaloid can tetrahydropapaverine, papaverine, rotundine, glaucine, or noscapine.

The “output signal” refers to any detectable signal that indicates the presence of the input signal. For example, the output signal can be the expression, or repression of expression, of a gene. The output signal can be fluorescence, luminescence, or a colorimetric signal. Examples include, but are not limited to, bioluminescent proteins such as a luciferase, a β-galactosidase, a lactamase, a horseradish peroxidase, an alkaline phosphatase, a β-glucuronidase or a β-glucosidase. Examples of luciferases include, but are not necessarily limited to, a Renilla luciferase, a Firefly luciferase, a Coelenterate luciferase, a North American glow worm luciferase, a click beetle luciferase, a railroad worm luciferase, a bacterial luciferase, a Gaussia luciferase, Aequorin, an Arachnocampa luciferase, or a biologically active variant or fragment of any one, or chimera of two or more, thereof. The output signal can be fluorescent. Examples include, but are not limited to, green fluorescent protein (GFP), blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Venus, mOrange, Topaz, GFPuv, destabilized EGFP (dEGFP), destabilized ECFP (dECFP), destabilised EYFP (dEYFP), HcRed, t-HcRed, DsRed, DsRed2, t-dimer2, t-dimer2 (12), mRFP1, pocilloporin, Renilla GFP, Monster GFP, paGFP, Kaede protein or a Phycobiliprotein, or a biologically active variant or fragment of any one thereof. The fluorescent molecule can also be a non-protein. Examples include, but are not necessarily limited to, an Alexa Fluor dye, Bodipy dye, Cy dye, fluorescein, dansyl, umbelliferone, fluorescent microsphere, luminescent microsphere, fluorescent nanocrystal, Marina Blue, Cascade Blue, Cascade Yellow, Pacific Blue, Oregon Green, Tetramethylrhodamine, Rhodamine, Texas Red, rare earth element chelates, or any combination or derivatives thereof.

The input signal can be converted to the output signal by a transduction system. The transduction system can comprise a transcriptional activator or transcriptional repressor of the output signal. For example, the transcriptional activator or transcriptional repressor is encoded with the engineered substrate promiscuous regulator. The transduction system can further comprise a promoter or operator and a regulator. Methods of using transduction systems in a biosensor are known to those of skill in the art and can be deployed with the method disclosed herein. Interaction between the input signal and the transduction system can be covalent or non-covalent.

### Cells and Plasmids Comprising Engineered Biosensors

The disclosed biosensors, systems, and methods may be utilized and/or performed using any suitable cell. For example, the biosensors disclosed herein can be integrated into a host genome, or can be in a plasmid. Disclosed herein is a host cell that produces one or more ligands, such as a BIA. Any convenient type of host cell may be utilized in producing the ligand, see, e.g., US2008/0176754, the disclosure of which is incorporated by reference in its entirety. Any convenient cells may be utilized in the subject host cells and methods. In some cases, the host cells are non-plant cells. In certain cases, the host cells are insect cells, mammalian cells, bacterial cells or yeast cells. Host cells of interest include, but are not limited to, bacterial cells, such as Bacillus subtilis, Escherichia coli, Streptomyces and Salmonella typhimuium cells and insect cells such as Drosophila melanogaster S2 and Spodoptera frugiperda Sf9 cells. In some embodiments, the host cells are yeast cells or E. coli cells. In certain embodiments, the yeast cells can be of the species Saccharomyces cerevisiae (S. cerevisiae).

The term “host cells,” as used herein, are cells that harbor one or more heterologous coding sequences which encode activity(ies) that enable the host cells to produce desired ligands e.g., as described herein. The heterologous coding sequences could be integrated stably into the genome of the host cells, or the heterologous coding sequences can be transiently inserted into the host cell. As used herein, the term “heterologous coding sequence” is used to indicate any polynucleotide that codes for, or ultimately codes for, a peptide or protein or its equivalent amino acid sequence, e.g., an enzyme, that is not normally present in the host organism and can be expressed in the host cell under proper conditions. As such, “heterologous coding sequences” includes multiple copies of coding sequences that are normally present in the host cell, such that the cell is expressing additional copies of a coding sequence that are not normally present in the cells. The heterologous coding sequences can be RNA or any type thereof, e.g., mRNA, DNA or any type thereof, e.g., cDNA, or a hybrid of RNA/DNA. Examples of coding sequences include, but are not limited to, full-length transcription units that comprise such features as the coding sequence, introns, promoter regions, 3′-UTRs and enhancer regions.

As used herein, the term “heterologous coding sequences” also includes the coding portion of the peptide or enzyme, i.e., the cDNA or mRNA sequence, of the peptide or enzyme, as well as the coding portion of the full-length transcriptional unit, i.e., the gene comprising introns and exons, as well as “codon optimized” sequences, truncated sequences or other forms of altered sequences that code for the enzyme or code for its equivalent amino acid sequence, provided that the equivalent amino acid sequence produces a functional protein. Such equivalent amino acid sequences can have a deletion of one or more amino acids, with the deletion being N-terminal, C-terminal or internal. Truncated forms are envisioned as long as they have the catalytic capability indicated herein. Fusions of two or more enzymes are also envisioned to facilitate the transfer of metabolites in the pathway, provided that catalytic activities are maintained.

Operable fragments, mutants or truncated forms may be identified by modeling and/or screening. This is made possible by deletion of, for example, N-terminal, C-terminal or internal regions of the protein in a step-wise fashion, followed by analysis of the resulting derivative with regard to its activity for the desired reaction compared to the original sequence. If the derivative in question operates in this capacity, it is considered to constitute an equivalent derivative of the enzyme proper.

The host cells may also be modified to possess one or more genetic alterations to accommodate the heterologous coding sequences. Alterations of the native host genome include, but are not limited to, modifying the genome to reduce or ablate expression of a specific protein that may interfere with the desired pathway. The presence of such native proteins may rapidly convert one of the intermediates or final products of the pathway into a metabolite or other compound that is not usable in the desired pathway. Thus, if the activity of the native enzyme were reduced or altogether absent, the produced intermediates would be more readily available for incorporation into the desired product.

Such gene deletions may lead to improved ligand production. The expression of cytochrome P450s may induce the unfolded protein response and may cause the ER to proliferate. Deletion of genes associated with these stress responses may control or reduce overall burden on the host cell and improve pathway performance. Genetic alterations may also include modifying the promoters of endogenous genes to increase expression and/or introducing additional copies of endogenous genes. Examples of this include the construction/use of strains which overexpress the endogenous yeast NADPH-P450 reductase CPR1 to increase activity of heterologous P450 enzymes. In addition, endogenous enzymes such as ARO8, 9, and 10, which are directly involved in the synthesis of intermediate metabolites, may also be overexpressed.

In some instances, the expression of each type of ligand is increased through additional gene copies (i.e., multiple copies), which increases intermediate accumulation and ultimately ligand production. Embodiments of the present invention include increased ligand production in a host cell through simultaneous expression of multiple species variants of a single or multiple enzymes. In some cases, additional gene copies of a single or multiple enzymes are included in the host cell. Any convenient methods may be utilized in including multiple copies of a heterologous coding sequence for an enzyme in the host cell.

In some embodiments, the host cell includes multiple copies of a heterologous coding sequence for an enzyme, such as 2 or more, 3 or more, 4 or more, 5 or more, or even 10 or more copies. In certain embodiments, the host cell include multiple copies of heterologous coding sequences for one or more enzymes, such as multiple copies of two or more, three or more, four or more, etc. In some cases, the multiple copies of the heterologous coding sequence for an enzyme are derived from two or more different source organisms as compared to the host cell. For example, the host cell may include multiple copies of one heterologous coding sequence, where each of the copies is derived from a different source organism. As such, each copy may include some variations in explicit sequences based on inter-species differences of the enzyme of interest that is encoded by the heterologous coding sequence.

### Methods of Engineering a Biosensor

Also disclosed herein is a method of engineering a substrate-promiscuous regulator to function as a biosensor, the method comprising identifying a naturally occurring substrate-promiscuous regulator; engineering the naturally occurring substrate-promiscuous regulator for increased sensitivity to an input signal when compared to the naturally occurring substrate promiscuous regulator; introducing into a cell a nucleic acid encoding the engineered substrate-promiscuous regulator, and a transduction system for providing an output signal, wherein said output signal is generated in response to interaction with the input signal; exposing the cell of step c) to the input signal; and detecting an output signal; wherein detection of said output signal indicates a functional biosensor.

Genetic engineering of a naturally occurring substrate-promiscuous regulator to be specific (or more specific) for a given ligand can be via genetic mutation of the naturally occurring substrate-promiscuous regulator. For example, this can occur through chip-based DNA synthesis, CRISPR, multiplexed genome engineering, in vivo mutagenesis, random mutagenesis, recombineering, or site-directed mutagenesis. The method can comprise determining a “hotspot” for potential input signal recognition and creating mutations within the hotspot to create an engineered substrate-promiscuous regulator. This ‘hotspot’ may include amino acid residues that are known or predicted to directly interact with the input signal. An example of this can be found in Example 1 with RamR, a transcription regulator found in Salmonella.

### Methods of Using a Biosensor

Also disclosed herein are methods of using the biosensors of the present invention. For example, Mehrotra et al. (J Oral Biol Craniofac Res. 2016 May-August; 6 (2): 153-159), (incorporated herein in its entirety for its disclosure regarding the uses of biosensors) discusses multiple ways that biosensors can be used, all of which are envisioned in the present invention. For example, biosensors can be used in food processing, monitoring, food authenticity, quality and safety. Biosensors can be used for the detection of pathogens in food. For example, the presence of Escherichia coli in vegetables, is a bioindicator of fecal contamination in food. Enzymatic biosensors are also employed in the dairy industry. The detection and quantification of food sweeteners is also envisioned.

Biosensors can also be used in fermentation processes. In fermentation industries, process safety and product quality are crucial. Thus effective monitoring of the fermentation process is imperative to develop, optimize and maintain biological reactors at maximum efficacy. Biosensors can be utilized to monitor the presence of products, biomass, enzyme, antibody or by-products of the process to indirectly measure the process conditions. Biosensors are also employed in ion exchange retrieval, where detection of change of biochemical composition is carried out.

Biosensors can also be used for sustainable food safety. The term food quality refers to the appearance, taste, smell, nutritional value, freshness, flavor, texture and chemicals. Smart monitoring of nutrients and fast screening of biological and chemical contaminants are of paramount importance when it comes to food quality and safety. Biosensors are being employed to perceive general toxicity and specific toxic metals, due to their capability to react with only the hazardous fractions of metal ions.

In the discipline of medical science, the applications of biosensors are very applicable. For example, glucose biosensors are widely used in clinical applications for diagnosis of diabetes mellitus, which requires precise control over blood-glucose levels. Biosensors are being used in the medical field to diagnose infectious diseases. The various other biosensors applications include: quantitative measurement of cardiac markers in undiluted serum, microfluidic impedance assay for controlling endothelin-induced cardiac hypertrophy, immunosensor array for clinical immunophenotyping of acute leukemias, effect of oxazaborolidines on immobilized fructosyltransferase in dental diseases; histone deacylase (HDAC) inhibitor assay from resonance energy transfer, biochip for a quick and accurate detection of multiple cancer markers and neurochemical detection by diamond microneedle electrodes. Biosensors can also be utilized to identify missing components pertinent to metabolism, regulation, or transport of an analyte.

Biosensors can be used in metabolic engineering. Environmental concerns and lack of sustainability of petroleum-derived products are gradually exhorting need for development of microbial cell factories for synthesis of chemicals. A substantial fraction of fuels, commodity chemicals and pharmaceuticals can be produced from renewable feedstocks by exploiting microorganisms rather than relying on petroleum refining or extraction from plants. The high capacity for diversity generation also requires efficient screening methods to select the individuals carrying the desired phenotype. The earlier methods were spectroscopy-based enzymatic assay analytics however they had limited throughput. To circumvent this obstacle genetically encoded biosensors that enable in vivo monitoring of cellular metabolism were developed which offered the ability for high-throughput screening and selection using fluorescence-activated cell sorting (FACS) and cell survival, respectively. This form of application also extends to the high-throughput engineering not only of whole cells, or microbial factories, but also for individual enzymes or groups of enzymes. These applications are especially relevant to the pharmaceutical industry, whereby millions of enzymes must be screened for improved activity on a target chemical.

### Kits and Proteins/Nucleic Acids

Also disclosed herein is a kit, wherein the kit comprises a biosensor comprising an engineered substrate-promiscuous regulator, wherein said substrate-promiscuous regulator has been engineered to interact more efficiently with an input signal (also referred to herein as a ligand, or target) than does the naturally occurring substrate promiscuous regulator; and an output signal; wherein said output signal is generated in response to interaction with the input signal. The kit disclosed herein can be customized to be specific for a given ligand, for example, or for a series of different ligands.

The kit can comprise a plasmid encoding the engineered biosensor, or a cell with these elements integrated within its genome. The cell can have the biosensor and corresponding elements needed for expression engineered into the cell, or, alternatively, the cell can be transformed with a plasmid. The kit can further comprise components needed for detection of expression of a target molecule, such as the individual biosensor proteins themselves. The protein sensors may be purified individually and used outside a cellular context. One of skill in the art will understand what components can be included in such a kit.

An engineered variant of RamR is disclosed herein. RamR comprises the sequence SEQ ID NO: 3. The engineered variant comprises SEQ ID NOs: 1-6, and is encoded by the nucleic acid SEQ ID NO: 7-12. Disclosed herein are functional variants of SEQ ID NOS: 1 and 2, such as those with 90, 91, 92, 93, 94, 95, 96, 97, 98, or 99% identity to SEQ ID NO: 1 or 2. For instance, disclosed are amino acids that vary from SEQ ID NO: 1 by 1, 2, 3, 4, 5, 6, 7, 8, 9, or 10 amino acids. Also disclosed are nucleic acids that vary from SEQ ID NO: 2 by 1 by 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. The differences can be due to additions, deletions, or substitutions of amino acids or nucleic acids.

SEQ ID NO 1: (GLAU4. This sensor binds to glaucine)
MVARPKSEDKKQALLEAATQAIAQSGIAASTAVIARNAGVAEGTLFRYFATKDELINTL
YLHLTQDWCQSIIMELDRSITDAKMMTRFLWNSWISWGLNHPARHRAIRQLAVSEKLT
KETEQRADDMFPELRDHLHRNVLMVFMSDEYRAFGDGLFLALAETTMDFAARDPARA
GEYIALGFEAMWRALAREEQ
SEQ ID NO 2: (NOS4. This sensor binds to noscapine)
MVARPKSEDKKQALLEAATQAIAQSGIAASTAVIARNAGVAEGTLFRYFATKDELINTL
YLHLTHDMCQSLIMELDRSITDAKMMTRFIWNSYISWGLNHPARHRAIRQLAVSEKLTK
ETRQRARDMFPELRDLCYRSLLMVFMSDEYRAFGDGLFMALAETTMDFAARDPARAG
EYIALGFEAMWRALTREEQ
SEQ ID NO 3: (PAP4. This sensor binds to papaverine)
MVARPKSEDKKQALLEAATQAIAQSGIAASTAVIARNAGVAEGTLFRYFATKDELINTL
YLHLRQDLCQSLIMELDRSITDAKMMMRFIWNSGISWGLNHPARHRAIRQLAVSEKLTK
ETHQRDLDMFPELRDILHRRVLMVFMSDEYRAFGDGLFLALAETTMDFAARDPARAGE
YIALGFEAMWRALTREEQ
SEQ ID NO 4: (ROTU4. This sensor binds to rotundine)
MVARPKSEDKKQALLEAATQAIAQSGIAASTAVIARNAGVAEGTLFRYFATKDELINTL
YLHLYQDHCQSLIMELDRSITDAKMMIRFTWNSYISWGLNHPARHRAIRQLAVSEKLTK
ETKQRIEDMFPELRDILHRSVLMVFMSDEYSAFGKGLFYALAETTMDFAARDPARAGE
YIALGFEAMWRALTREEQ
SEQ ID NO 5: (THP4. This sensor binds to tetrahydropapaverine)
MVARPKSEDKKQALLEAATQAIAQSGTAASTAVIARNAGVAEGTLFRYFATKDELINTL
YLHLFQDWCQSSIMELDRSITDAKMMTRFLWNSIISWGLNHPARHRAIRQLAVSEKLSK
ETVQRADDMFPELRDIVHREVLMVFMSDEYRAFGEGLFLALAETTMDFAARDPARAGE
YIALGFEAMWRALTREEQ
SEQ ID NO 6: (4NB2. This sensor binds to 4-Omethylnorbelladine)
MVARPKSEDKKQALLEAATQAIAQSGIAASTAVIARNAGVAEGTLFRYFATKDELINTL
YLHLTQDMCQSMIMELDRSITDAKMMTRFIWNSYISWGLNHPARHRAIRQLAVSEKLT
KETEQRADDMFPELRDLDHRGVLMVFMSDEYRAFGDGLFLALAETTMDFAARDPARA
GEYIALGFEAMWRALTREEQ
SEQ ID NO 7: (DNA sequences for GLAU4)
ATGGTTGCTCGCCCAAAGTCTGAGGACAAAAAGCAGGCATTGCTTGAAGCGGCAAC
TCAAGCCATCGCGCAATCAGGCATTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGACCCAGGACTGGTGCCAATCAATCATCATGGAATTGG
ATCGTTCTATTACTGACGCTAAGATGATGACCCGTTTTTTGTGGAACAGTTGGATTA
GCTGGGGATTGAACCACCCAGCTCGCCATCGTGCCATTCGTCAGTTGGCGGTTTCTG
AAAAGTTGACGAAGGAAACCGAACAACGCGCGGATGATATGTTCCCGGAGTTACGC
GACCACCTGCACCGTAACGTTCTTATGGTGTTTATGTCCGACGAGTACCGCGCCTTC
GGCGACGGGTTGTTCTTGGCGCTTGCTGAGACGACTATGGATTTCGCTGCGCGCGAC
CCGGCTCGCGCTGGTGAGTACATTGCGTTGGGCTTCGAGGCTATGTGGCGCGCATTG
GCCCGCGAAGAGCAGTAA
SEQ ID NO 8: (DNA sequences for NOS4)
ATGGTTGCTCGCCCAAAGTCTGAGGACAAAAAGCAGGCATTGCTTGAAGCGGCAAC
TCAAGCCATCGCGCAATCAGGCATTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGACCCATGACATGTGCCAATCACTGATCATGGAATTGG
ATCGTTCTATTACTGACGCTAAGATGATGACCCGTTTTATCTGGAACAGTTATATTA
GCTGGGGATTGAACCACCCAGCTCGCCATCGTGCCATTCGTCAGTTGGCGGTTTCTG
AAAAGTTGACGAAGGAAACCCGCCAACGCGCCCGCGATATGTTCCCGGAGTTACGC
GACTTGTGCTACCGTAGTTTGCTTATGGTGTTTATGTCCGACGAGTACCGCGCCTTCG
GCGACGGGTTGTTCATGGCGCTTGCTGAGACGACTATGGATTTCGCTGCGCGCGACC
CGGCTCGCGCTGGTGAGTACATTGCGTTGGGCTTCGAAGCTATGTGGCGCGCACTTA
CGCGCGAAGAGCAGTAA
SEQ ID NO 9: (DNA sequences for PAP4)
ATGGTTGCTCGCCCAAAGTCTGAGGACAAAAAGCAGGCATTGCTTGAAGCGGCAAC
TCAAGCCATCGCGCAATCAGGCATTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGAGGCAGGACCTGTGCCAATCACTCATCATGGAATTGG
ATCGTTCTATTACTGACGCTAAGATGATGATGCGTTTTATCTGGAACAGTGGCATTA
GCTGGGGATTGAACCACCCAGCTCGCCATCGTGCCATTCGTCAGTTGGCGGTTTCTG
AAAAGTTGACGAAGGAAACCCACCAACGCGACCTGGATATGTTCCCGGAGTTACGC
GACATCCTGCACCGTAGGGTTCTTATGGTGTTTATGTCCGACGAGTACCGCGCCTTC
GGCGACGGGTTGTTCTTGGCGCTTGCTGAGACGACTATGGATTTCGCTGCGCGCGAC
CCGGCTCGCGCTGGTGAGTACATTGCGTTGGGCTTCGAGGCTATGTGGCGCGCACTT
ACGCGCGAAGAGCAGTAA
SEQ ID NO 10: (DNA sequences for ROTU4)
ATGGTTGCTCGCCCAAAGTCTGAGGACAAAAAGCAGGCATTGCTTGAAGCGGCAAC
TCAAGCCATCGCGCAATCAGGCATTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGTACCAGGACCACTGCCAATCACTGATCATGGAATTGG
ATCGTTCTATTACTGACGCTAAGATGATGATCCGTTTTACCTGGAACAGTTACATTA
GCTGGGGATTGAACCACCCAGCTCGCCATCGTGCCATTCGTCAGTTGGCGGTTTCTG
AAAAGTTGACGAAGGAAACCAAGCAACGCATCGAGGATATGTTCCCGGAGTTACGC
GACATCCTGCACCGTAGTGTTCTTATGGTGTTTATGTCCGACGAGTACTCCGCCTTCG
GCAAGGGGTTGTTCTACGCGCTTGCTGAGACGACTATGGATTTCGCTGCGCGCGACC
CGGCTCGCGCTGGTGAGTACATTGCGTTGGGCTTCGAGGCTATGTGGCGCGCACTTA
CGCGCGAAGAGCAGTAA
SEQ ID NO 11: (DNA sequences for THP4)
ATGGTTGCTCGCCCAAAGTCTGAGGACAAAAAGCAGGCATTGCTTGAAGCAGCAAC
TCAAGCCATCGCGCAATCAGGCACTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGTTCCAGGACTGGTGCCAATCATCCATCATGGAATTGG
ATCGTTCTATTACTGACGCTAAGATGATGACGCGTTTTCTCTGGAACAGTATCATTA
GCTGGGGATTAAACCACCCAGCTCGCCATCGTGCCATTCGTCAGTTGGCGGTTTCTG
AAAAGTTGTCGAAGGAGACCGTACAACGCGCGGATGATATGTTCCCGGAGTTACGC
GACATCGTCCACCGTGAGGTTCTTATGGTGTTTATGTCCGACGAGTACCGCGCCTTC
GGCGAAGGGTTGTTCTTGGCGCTTGCTGAGACGACTATGGATTTCGCTGCGCGCGAC
CCGGCTCGCGCTGGTGAGTACATTGCGTTGGGCTTCGAGGCTATGTGGCGCGCACTT
ACGCGCGAAGAGCAGTAA
SEQ ID NO 12: (DNA sequences for 4NB2)
ATGGTTGCTCGCCCAAAGTCTGAGGACAAAAAGCAGGCATTGCTTGAAGCGGCAAC
TCAAGCCATCGCGCAATCAGGCATTGCCGCTAGTACCGCTGTAATTGCACGCAATGC
GGGAGTTGCGGAAGGGACGTTGTTCCGCTATTTCGCAACGAAAGATGAGTTGATCA
ACACCCTTTACTTACATTTGACCCAGGACATGTGCCAATCAATGATCATGGAATTGG
ATCGTTCTATTACTGACGCTAAGATGATGACCCGTTTTATCTGGAACAGTTATATTA
GCTGGGGATTGAACCACCCAGCTCGCCATCGTGCCATTCGTCAGTTGGCGGTTTCTG
AAAAGTTGACGAAGGAAACCGAACAACGCGCGGATGATATGTTCCCGGAGTTACGC
GACCTCGACCACCGTGGCGTTCTTATGGTGTTTATGTCCGACGAGTACCGCGCCTTC
GGCGACGGGTTGTTCTTGGCGCTTGCTGAGACGACTATGGATTTCGCTGCGCGCGAC
CCGGCTCGCGCTGGTGAGTACATTGCGTTGGGCTTCGAGGCTATGTGGCGCGCACTT
ACGCGCGAAGAGCAGTAA

### EXAMPLES

### Example 1: Using Fungible Biosensors to Evolve Improved Alkaloid Biosynthesis

In the past decade microbial engineering for production of complex therapeutic plant metabolites has significantly advanced. However, a key bottleneck in the engineering process is screening to identify variants with improved activity, which is typically performed using low-throughput chromatography-based methods. Genetic biosensors can overcome this limitation and increase throughput by several orders of magnitude, but few biosensors exist in Nature for plant metabolites with therapeutic potential. This gap is addressed by synergizing the extreme promiscuity of a multidrug resistance regulator, RamR from Salmonella typhimurium, with a custom directed evolution circuit architecture to create a series of highly specific biosensors for the plant alkaloids tetrahydropapaverine, papaverine, glaucine, rotundine, and noscapine. High resolution structures of evolved biosensors elucidate key adaptations acquired during evolutionary specialization. We subsequently apply one biosensor to evolve a plant methyltransferase, enabling the microbial production of tetrahydropapaverine, an immediate precursor to four modern pharmaceuticals. Biosensor generalists can be rapidly evolved for therapeutic plant metabolites and enable high-throughput pathway engineering.

Disclosed herein are methods of exploiting a key insight from natural selection, that a protein's substrate promiscuity correlates with its evolvability [10]. Thus, by starting with biosensors that are broadly represented in phylogeny, and whose substrate specificities have already been shown to be fungible in terms of natural ligands, it should be possible to create biosensors for virtually any compound. In particular, prokaryotic multidrug resistance regulators, typically studied as mediators of broad-spectrum antibiotic resistance, have large substrate binding pockets and are known to recognize a raft of structurally-diverse lipophilic molecules via non-specific interactions [13]. Early studies suggest that they may also be highly evolvable; notably, just a single point mutation enabled one of these regulators, TtgR, to adopt substantial affinity for the non-cognate ligand resveratrol [14].

Using a novel directed evolution architecture that relies on both screening and selection, sensor libraries of over 105 members can be filtered into just a few high performing variants in under one week. As proof, a single multidrug resistance regulator, RamR from Salmonella typhimurium, was evolved to sensitively and specifically recognize five diverse therapeutic alkaloids. The high resolution structure of these sensors reveal how the malleable effector binding site can learn to specifically interact with entirely new ligands in wildly different ways.

### Results

### Identifying a BIA-Responsive Multidrug Resistance Regulator

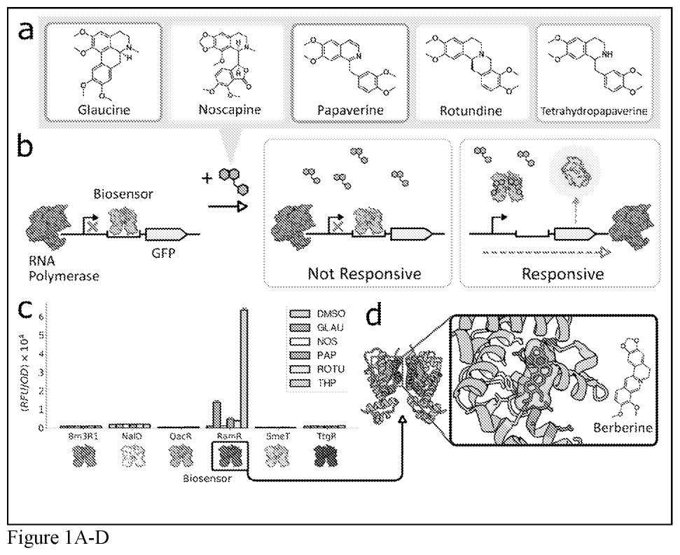

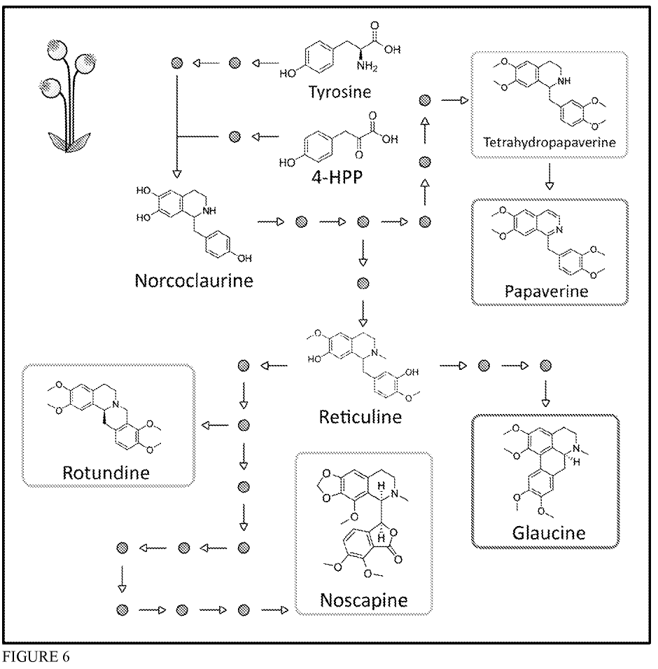

Given that therapeutic plant metabolites are largely lipophilic, it was reasoned that multidrug resistance regulators may display a modest affinity towards these compounds. Among plant-based therapeutics, we focused on generating sensors for benzylisoquinoline alkaloids (BIAs) since they (1) are rich in therapeutic activity, (2) have largely resolved biosynthetic pathways, and (3) are the subject of ongoing academic and commercial efforts [3,4]. Specifically, the five BIAs tetrahydropapaverine (THP), papaverine (PAP), rotundine (ROTU), glaucine (GLAU), and noscapine (NOS) were targeted, since these compounds are therapeutically relevant, commercially available, and belong to the structurally distinct benzylisoquinoline (THP and PAP), protoberberine, aporphine, and phthalideisoquinoline BIA families, respectively (FIG. 1a) (FIG. 6). Furthermore, the complete microbial biosynthesis of noscapine and rotundine have recently been reported [16, 17].

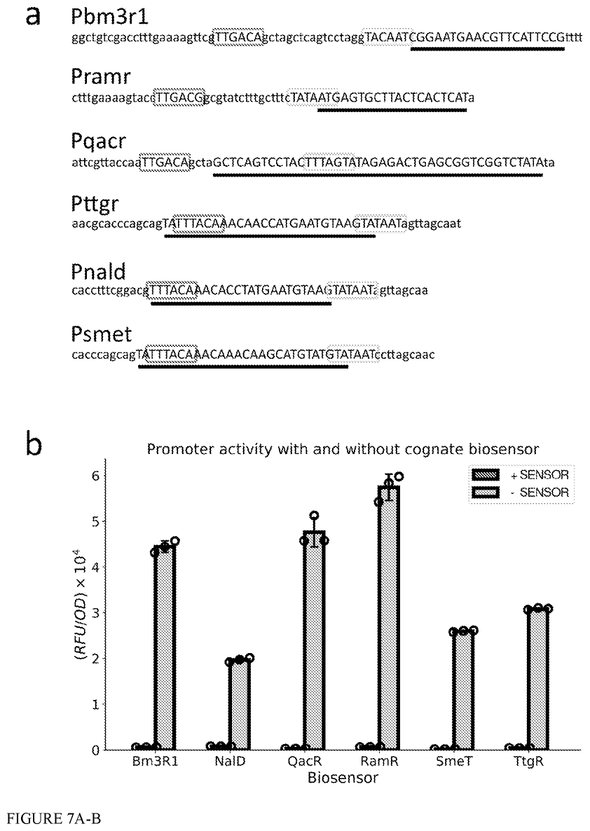

To identify a biosensor with some degree of BIA affinity to serve as a suitable scaffold for evolution, the responsiveness of six well characterized multidrug resistance regulators, QacR, TtgR, RamR, SmeT, NalD, and Bm3R1 to the target BIAs were assayed. Regulators were constitutively expressed on one plasmid (pReg) that was co-transformed with another plasmid bearing the regulator's cognate promoter expressing sfGFP (pGFP). Promoters for QacR and TtgR were obtained from the literature [18, 14] while promoters for the remainder were designed by either placing the sensor's operator downstream a medium strength promoter (Bm3R1) or by modifying the −35 or −10 regions of the sensor's native promoter towards the E. coli consensus (NalD, SmeT, RamR) [18*, 18**] if necessary to produce sufficient transcription (FIG. 7a). The ability of each regulator to repress transcription was confirmed by measuring the promoter activity with and without the expression of the cognate sensor via fluorescence (FIG. 7b). Upon screening, one sensor, RamR from S. typhimurium, was found to be moderately responsive to most target BIAs and was selected as the template for sensor evolution (FIG. 1c). The structure of RamR had been solved in complex with berberine (PDB: 3VW2), an alkaloid related to our target ligands, and was used to guide library design [19]. To generate sensor diversity for subsequent evolution, semi-rational libraries were created by simultaneously site-saturating three residues on five separate helices facing the ligand binding pocket (FIG. 1d). In addition, error-prone libraries of the entire coding sequence were generated with an average of two mutations relative to the template.

### Circuit Design for Biosensor Evolution

Transforming one promiscuous regulator into several highly specific alkaloid biosensors was expected to require extensive engineering, warranting a new approach to sensor design. Typically, biosensors are evolved by screening sensor libraries for low fluorescence in the absence of the target ligand and for high fluorescence in the presence of the target ligand, via fluorescence activated cell sorting (FACS). This approach however, suffers numerous drawbacks, including poor enrichment of sensors with a low background signal, the requirement for an expensive instrument and extensive training, and slow and laborious protocols since multiple independent rounds of sorting and counter-sorting are typically required prior to recovering clonal isolates. Therefore, a new directed evolution circuit architecture tailored for sensor evolution was designed, which is termed Seamless Enrichment of Ligand Inducible Sensors (SELIS), that amalgamated these steps and could quickly filter large libraries.

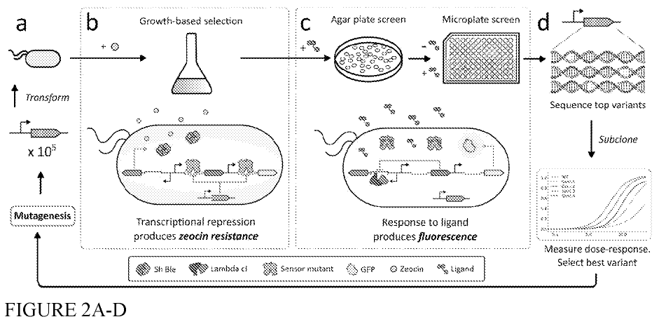

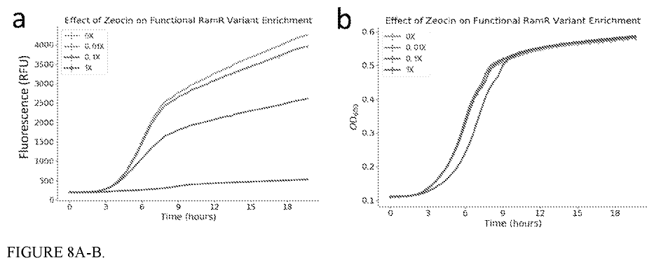

Three essential filtering steps are required for biosensor engineering; (1) removing sensors with a reduced ability to repress transcription in the absence of the target ligand, (2) removing variants that are responsive to non-target ligands, and (3) enriching variants that are more responsive to the target ligand. To implement the first two functions, the output of the sensor was inverted, via repression of the Lambda cl repressor, to express the zeocin resistance protein encoded by the Sh ble gene (FIG. 2b). In effect, cells containing inactive biosensors remain sensitive to the antibiotic zeocin due to continued repression of the Sh ble gene and are eliminated from the population, whereby cells with actively repressing sensors produce Sh ble and survive. Sh ble was chosen for its non-catalytic mechanism of action, enabling more titratable selection stringency [20]. Trial selections showed enrichment for functionally repressing RamR variants in a zeocin-dependent manner (FIG. 8). Non-target ligands can also be supplemented at this stage to counter select against non-specific sensors. Stringency for repression can be tuned by modifying the strength of the promoter expressing the sensor; a weaker promoter selects for variants that repress stronger.

To enrich variants that derepress in the presence of the target ligand, the output of the sensor was linked to the expression of GFP (FIG. 2c). Liquid cultures grown in the presence of zeocin are plated onto solid media containing the target ligand, but lacking zeocin. Highly fluorescent clones are isolated and re-phenotyped in liquid medium in both the presence and absence of the target ligand to determine the signal/noise ratio of each sensor variant. The stringency of this enrichment can be tuned by altering the amount of the target ligand applied to the solid media. Variants with low background and a high signal/noise ratio are sequenced and unique variants are then subcloned into a new vector and characterized using a wide range of ligand concentrations (FIG. 2d). The highest performing biosensor variant is then used as the template for the next round of evolution.

Using this circuit, which was named pSelis, a library containing ˜105 variants can be deconvoluted to yield phenotype and genotype data for high performing clones in just one week, without the need for specialized equipment. The SELIS methodology is broadly applicable to evolve virtually any prokaryotic ligand-inducible repressor.

### Evolving RamR Specificity Towards Benzylisoquinoline Alkaloids

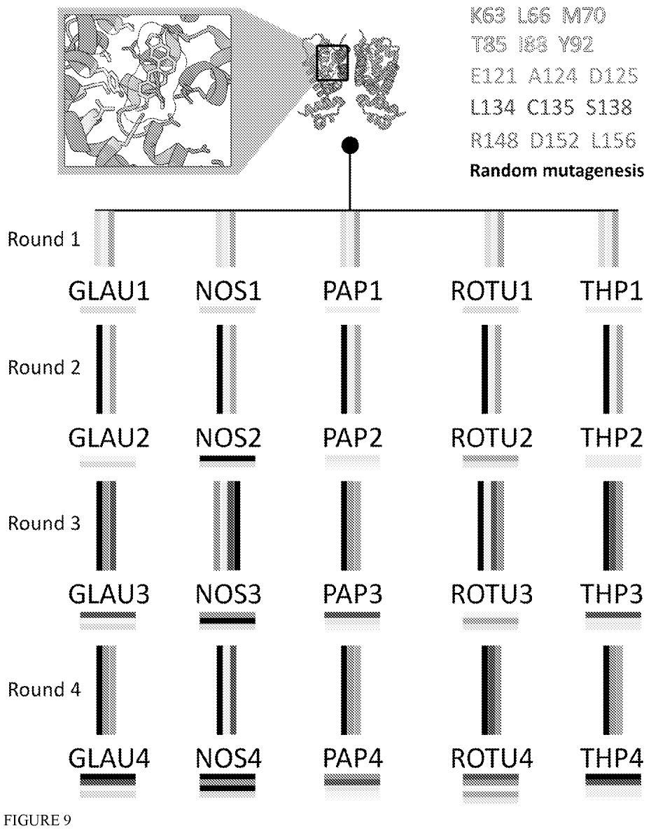

Multidrug resistance regulators are known to recognize structurally diverse ligands, however, the limits of their plasticity remains unexplored. For practical utility in microbial engineering projects, sensors must be both highly sensitive and highly specific for their target molecule to report on low-activity pathways and avoid false positives, respectively. Using wild-type RamR as the starting point, four rounds of evolution were performed for each evolutionary lineage towards one of five BIAs to create a total of 20 RamR sensor generations. As library positions fixed, new site-saturation libraries were included to reintroduce diversity (FIG. 9). Following the first round of evolution, the strength of the promoter expressing the RamR variant and the concentration of the target BIA were conditionally reduced to increase the selection stringency for repression and ligand responsiveness, respectively (Table 1). After the second round of evolution, 100 μM of all non-target BIAs were added during the growth-based selection to eliminate polyspecific sensor variants.

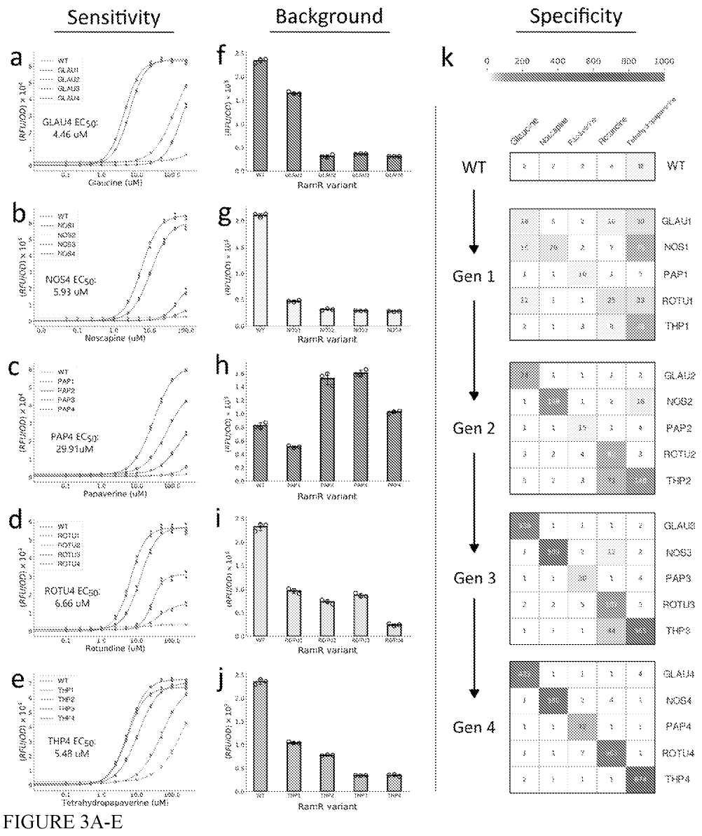

Over the course of four generations of evolution, discrete evolutionary lineages became highly sensitive to their cognate BIA. High sensitivity is a crucial property for practical application of biosensors for plant-derived therapeutics since initial product titers from recombinant hosts are expected to be extremely low. Despite having a barely detectable response to most target BIAs initially, four of the five final RamR variants had an EC₅₀ value under 7 μM, highlighting the plasticity of this biosensor scaffold (FIG. 3a-e). Notably, the detectable concentration range for the final noscapine biosensor is well within the reported level produced de novo in yeast [16]. Intermediate sensor variants produced throughout evolution cover a range of EC₅₀ values that may aid screening projects as a biosynthetic pathway improves. In addition, the background signal was also reduced to less than 40% of wild-type RamR for four of the five final biosensors (FIG. 3f-j). A low background signal typically correlates with an increased signal-to-noise ratio and reduced limit of detection.

Despite starting from the same generalist template, all five final biosensor variants are extremely specific for their matching BIA. High specificity is crucial for sensors used in strain engineering to avoid false positives arising from cross-reactivity with non-cognate ligands, particularly biosynthetic precursors. The final sensors display >100-fold preference for their cognate BIA over all other non-cognate BIAs when a solubility-limiting concentration (100 μM) of each compound was applied (FIG. 3K).

### Structures Reveal Shared and Unique Adaptations to Diverse Alkaloids

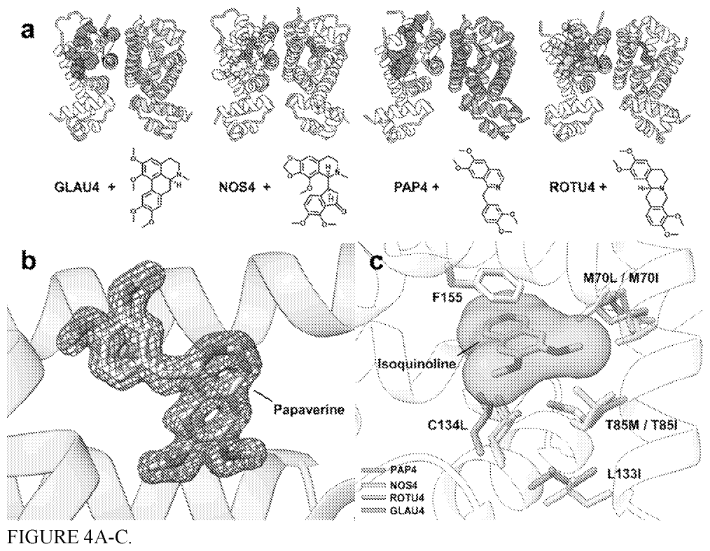

Since both the ligand sensitivity and specificity of RamR were dramatically transformed throughout evolution better understanding of the molecular adaptations employed was sought. Each evolved BIA sensor accumulated nine to thirteen mutations, which would be difficult to be explained with intuition or computational modeling. Therefore, the structures of four of the five evolved sensors was solved in complex with their cognate BIA: PAP4 with papaverine (1.6 Å), ROTU4 with rotundine (1.8 Å), GLAU4 with glaucine (2.0 Å), and NOS4 with noscapine (2.2 Å) (Table 2). The overall folding and dimerization of the evolved variants is highly identical to that of wild type RamR (FIG. 4a). A strong positive electron density was consistently detected at the binding site for each molecule in the asymmetric unit, which perfectly fit with the BIA chemical structures. (FIG. 4b).

BIAs are composed of heterocycle isoquinoline moiety and a benzyl group moiety, and how two ring components are interconnected distinguishes each BIA from others. Interestingly, the configuration of each ligand complexed with RamR variants reveals that one of the ring components is always ‘fixed’ underneath Phe155 due to π-π stacking interaction, while alternative moieties occupy different regions of the binding cavity. Moreover, the ring component parallel to Phe155 is recognized by a hydrophobic pocket formed by mutations in residue 70, 85, 133, and 134 (FIG. 4c). Specifically, C134 is consistently mutated into leucine to form a hydrophobic interaction with one of the ring components. Another mutation consistent in all variants is the mutation of M70 into a shorter hydrophobic residue (leucine or isoleucine), which reinforces hydrophobic interaction with the BIA ligand. The L133I substitution epistatically interacts with the residue at position 85 (PAP4: T85M/L133I; ROTU4: T851/L133I), where the less extended isoleucine side chain makes room for the bulkier mutation of T85 with higher hydrophobicity. Identification of this common binding pattern and key residues involved in BIA recognition can facilitate structure-guided engineering of sensors for morphinans and other therapeutic alkaloids.

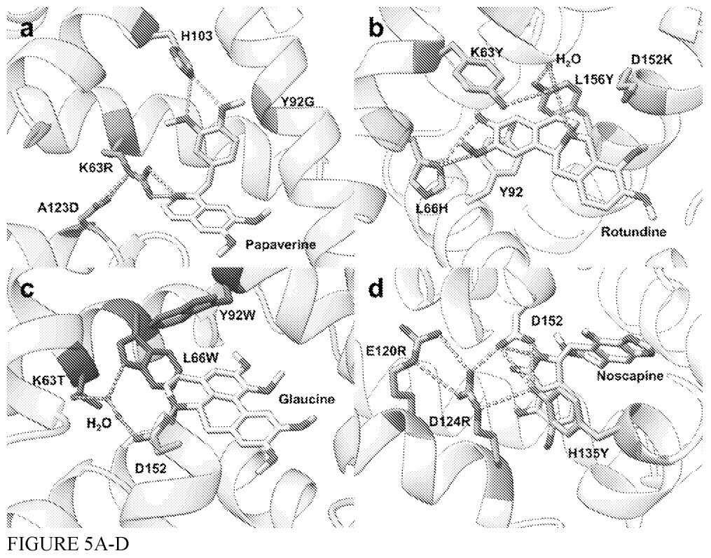

Despite the structural similarities among BIA ligands, each BIA biosensor employs unique mechanisms to accommodate heteroatoms and extra ring moiety that are not recognized by the common hydrophobic binding pattern mentioned above. Notably, the nitrogen atom of papaverine is coordinated by the K63R substitution of PAP4, which is strongly anchored by the adjacent A123D substitution (FIG. 5a). In addition, a] Y92G mutation creates a cavity allowing the occupancy by the dimethoxybenzyl group of papaverine (FIG. 5a). In ROTU4, The K63Y and L156Y substitutions coordinate two ordered water molecules to interact with the nitrogen atom of rotundine (FIG. 5b). The L66H substitution provides additional hydrophilic interaction with oxygen atoms of rotundine. Moreover, together with native Y92, the K63Y and L156Y mutations form a triple-tyrosine ‘hydrophobic cage’ that traps the dimethoxybenzyl group of rotundine (FIG. 5b). The L66W and Y92W substitutions in GLAU4 create a large tryptophan sandwich motif which pins the hydrophobic glaucine fused rings, while the native D152 residue interacts with glaucine's nitrogen atom (FIG. 5c). Finally, unlike other BIA biosensors, noscapine extends into a side pocket close to the active site for its specificity. The ester group of noscapine interacts with native D152, which ‘masks’ the nitrogen atom of noscapine from hydrophilic residues of RamR. The H135Y substitution assists the accommodation of dimethoxybenzyl moiety by forming pseudo π-π interaction and participating into the hydrogen bond network associated with the ester group of noscapine (FIG. 5d). Additionally, the mutation of E120 and D124 into highly flexible arginine residue creates an electrophilic network with H135Y and D152 to form favorable hydrophilic interaction with Noscapine (FIG. 5d). Interestingly, though all alkaloids exhibit similar orientation of the nitrogen atom of the ligands, each RamR variant employed a unique adaptation to stabilize it (FIG. 5a-d). These structural data highlight the inherent flexibility of the RamR protein to rapidly evolve new ligand specificity, suggesting that it is indeed a “privileged template” for biosensor engineering.

### DISCUSSION

Using a custom directed evolution architecture it was demonstrated that fungible biosensors can rapidly adapt to specifically and sensitively recognize therapeutic alkaloids, for which no extant biosensors exist. High resolution structures reveal that a single effector binding site employs disparate evolutionary avenues for increasing ligand affinity. Evolved sensors should provide practical utility for screening low-flux recombinant pathway activity in microbial hosts. As biocatalyst engineering projects become increasingly ambitious, by reconstituting long pathways in microbial hosts or evolving enzyme cascades for pharmaceutical synthesis [26], there is an increased reliance on high-throughput screening capabilities. The approach described herein should prove effective to address the growing demands for rapid chemical measurement.

The methodology presented expands the chemical space accessible to biosensors. In previous work, biosensors have been evolved to recognize ligands that are structurally related to the sensor's cognate ligand. This approach, however, is limited to chemicals, or analog thereof, for which a sensor in nature exists, which is exceedingly small. This approach to biosensor evolution is inspired by the mechanisms of natural selection: start with a generalist, and evolve to a specialist [10]. This avenue not only affords a wider chemical search space, but also bypasses the commonly observed process of evolving a specialist for the native ligand to a generalist before producing a specialist for the desired ligand.

These findings show that the ‘promiscuity-focused’ approach is generalizable to other ligands for which no natural sensor exists. For example, the original RamR template displayed a slight response towards many of the target alkaloids, which was substantially improved in four rounds of evolution. Therefore, even a minimal response to the target ligand indicates potential to develop a highly sensitive and selective biosensor. These observations are reminiscent of laboratory evolution studies with highly promiscuous enzymes [11, 12]. Furthermore, since BIAs are not intimately relevant to Salmonella typhimurium metabolism and RamR is known to recognize a range of steroids and nitrogen-containing aromatic compounds [19, 28], this approach is likely generalizable to other lipophilic plant natural products or even synthetic compounds. Implementation requirements include (1) the target analyte being able to cross the cell membrane, (2) the analyte not being prohibitively toxic to the host cell, and (3) the identification of a generalist sensor with some basal responsiveness to the analyte.

Structural data of evolved RamR variants should aid future efforts to engineer RamR towards other ligands. A common binding pattern and key residues involved in isoquinoline recognition, a privileged scaffold found in numerous benzylisoquinoline alkaloids, amaryllidaceae alkaloids, and synthetic pharmaceuticals were found. This structural data can inform intelligent library design for subsequent projects evolving RamR for ligands bearing the isoquinoline moiety, or even related groups, such as the quinoline and indole moieties abundant in natural and synthetic pharmaceuticals [30].

Novel biosensors engineered using this approach can seamlessly integrate with existing technologies to provide broader utility to the biotechnology community. Beyond their utility in high-throughput screening, biosensors have been used in dynamic regulatory schemes to improve production strain fitness and extend productivity lifetime [31, 32], as well as diagnostics for monitoring patient health and environmental sampling [33, 34]. Engineered sensors can also be paired with recently described genetic circuitry to reduce the limit of detection or improve the signal/noise ratio [35, 36, 37]. Furthermore, having a simple ‘roadblocking’ regulatory mechanism, repressor-based biosensors evolved in E. coli may likely function in a wide range of medically and industrially relevant hosts, such as yeasts, mammalian cells, and plants [38, 39, 40].

The genetic tools and paradigms reported here can serve as a platform for developing custom biosensors integral to future strain engineering endeavors.

### Methods

### Strains, Plasmids, and Media

E. coli DH10B (New England BioLabs, Ipswich, MA, USA) was used for all routine cloning and directed evolution. All biosensor systems were characterized in E. coli DH10B. E. coli BL21 DE3 (New England BioLabs, Ipswich, MA, USA) was used for protein expression. LB-Miller (LB) media (BD, Franklin Lakes, NJ, USA) was used for routine cloning, fluorescence assays, directed evolution, and orthogonality assays unless specifically noted. Terrific broth (TB) (Thermo Fisher Scientific, CAT #: 22711022) was used for protein purification. LB+1.5% agar (BD, Franklin Lakes, NJ, USA) plates were used for routine cloning and directed evolution. The plasmids described in this work were constructed using Gibson assembly and standard molecular biology techniques. Synthetic genes, obtained as gBlocks, and primers were purchased from IDT. Relevant plasmid sequences are provided herein and those for final alkaloid sensors are available through Addgene. The pSelis plasmid can be requested from the corresponding authors.

### Benzylisoquinoline Alkaloids

Cells were induced with the following chemicals: norlaudanosoline (NOR) (HDH Pharma Inc. CAT #: 29030); tetrahydropapaverine (THP) (Tokyo Chemical Company, product #: N0918); papaverine (PAP) (MP Biomedicals LLC. CAT #: 190261); glaucine (GLAU) (Carbosynth Ltd. product #: FG137572); rotundine (ROTU) (Alfa Aesar, product #: J63328); noscapine (NOS) (Aldrich, SKU: 363960-5G); norreticuline (NRT) (Selena Chem Ltd. product #: CSC000735172).

### Chemical Transformation

For routine transformations, strains were made competent for chemical transformation. 5 mL of an overnight culture of DH10B cells were subcultured into 500 mL of LB media and grows at 37° C., 250 r.p.m. for 3 h. Cultures were centrifuged (3,500 g, 4° C., 10 min), and pellets were washed in 70 mL of chemical competence buffer (10% glycerol, 100 mM CaCl2) and centrifuged again (3,500 g, 4° C., 10 min). The resulting pellets were resuspended in 20 mL of chemical competence buffer. After 30 minutes on ice, cells were divided into 250 μL aliquots and flash frozen in liquid nitrogen. Competent cells were stored at −80° C. until use.

### Promoter Design and Biosensor Response Assay

Promoters for TtgR and QacR were derived from the literature [18, 14]. For the RamR promoter, a region 60 base pairs upstream the known operator sequence as well as the operator itself was extracted from the Salmonella typhimurium genome (WP_000113609.1). NalD and SmeT are homologs of TtgR, therefore modifications from the Pttgr promoter were made to match the sequence of the NalD operator [18*] and SmeT operator [18**]. For the Pbm3r1, the known Bm3R1 operator was placed immediately after the −10 region of a synthetic medium strength promoter. All promoter sequences are listed in FIG. 7. The pReg and pGFP equivalents for each regulator were co-transformed into DH10B cells and plated on an LB agar plate with appropriate antibiotics. Three separate colonies were picked for each transformation and were grown overnight. The following day, 20 uL of each culture was then used to inoculate six separate wells within a 2 mL 96-deep-well plate (Corning, Product #: P-DW-20-C-S) sealed with an AeraSeal film (Excel Scientific, Victorville, CA, USA) containing 900 μL of LB media, one for each test ligand and a solvent control. After 2 hours of growth at 37° C. cultures were induced with 100 μL of LB media containing either 10 μL of DMSO or 100 μL of LB media containing one of the five target BIAs dissolved in 10 μL of DMSO. Cultures were grown for an additional 4 hours at 37° C., 250 r.p.m and subsequently centrifuged (3,500 g, 4° C., 10 min). Supernatant was removed and cell pellets were resuspended in 1 mL of PBS (137 mM NaCl, 2.7 mM KCl, 10 mM Na2HPO4, 1.8 mM KH2PO4. pH 7.4). 100 μL of the cell resuspension for each condition was transferred to a 96 well microtiter plate (Corning, Product #: 3904), from which the fluorescence (Ex: 485 nM, Em: 509 nM) and absorbance (600 nM) was measured using the Tecan Infinite M1000 plate reader.

### RamR Library Design and Construction

Five semi-rational libraries were designed, each targeting three inward-facing residues on one of five helices of the RamR ligand binding pocket (FIG. 1d). Libraries were generated using overlap PCR with redundant NNS codons using Accuprime Pfx (Thermo Fisher, CAT #: 12344024) and cloned into pReg. E. coli DH10B bearing pSelis was transformed with the resulting library. Transformation efficiency always exceeded 106 for each round of selection, indicating several fold coverage of the library. Transformed cells were grown in LB media overnight at 37° C. in carbenicillin and chloramphenicol.

### Directed Evolution of RamR Biosensors

Twenty uL of cell culture bearing the sensor library was seeded into 5 mL of fresh LB containing appropriate antibiotics, 100 ug/mL zeocin (Thermo Fisher. CAT #: R25001), and 100 uM of non-target BIAs (for rounds three and four) and were grown at 37° C. for seven hours. Following incubation, 0.5 uL of culture was diluted into 1 mL of LB media, from which 100 uL was further diluted into 900 μL of LB media. 300 μL of this mixture was then plated across three LB agar plates containing carbenicillin, chloramphenicol and the target BIA dissolved in DMSO. Plates were incubated overnight at 37° C. The following day the brightest colonies were picked and grown overnight in 1 mL of LB media containing appropriate antibiotics within a 96-deep-well plate sealed with an AeraSeal film at 37° C. A glycerol stock of cells containing pSelis and pReg bearing the parental RamR variant was also inoculated in 5 mL of LB for overnight growth.

The following day, 20 μL of each culture was used to inoculate two separate wells within a new 96-deep-well plate containing 900 μL of LB media. Additionally, eight separate wells containing 1 mL of LB media were inoculated with 20 μL L of the overnight culture expressing the parental RamR variant. A typical arrangement would have 44 unique clones on the top half of the plate, duplicates of those clones on the bottom half of the plate, and the right-most column occupied by cells harboring the parental RamR variant. After 2 hours of growth at 37° C. the top half of the 96-well plate was induced with 100 μL of LB media containing 10 μL of DMSO whereas the bottom half of the plate was induced with 100 μL of LB media containing the target BIA dissolved in 10 μL of DMSO. The concentration of BIA used for induction is typically the same concentration used in the LB agar plate for screening during that particular round of evolution. Cultures were grown for an additional 4 hours at 37° C., 250 r.p.m and subsequently centrifuged (3,500 g, 4° C., 10 min). Supernatant was removed and cell pellets were resuspended in 1 mL of PBS. 100 μL of the cell resuspension for each condition was transferred to a 96 well microtiter plate, from which the fluorescence (Ex: 485 nM, Em: 509 nM) and absorbance (600 nM) was measured using the Tecan Infinite M1000. Clones with the highest signal-to-noise ratio were then sequenced and subcloned into a fresh pReg vector.

For sensor variant validation, the subcloned pReg vectors expressing the sensor variants were transformed into DH10B cells bearing pGFP. These cultures were then assayed, as described “Response function measurements” using eight different concentrations of the target BIA. Sensor variants that displayed a combination of a low background, a reduced EC₅₀ for the target BIA, and a high signal/noise ratio were used as templates for the next round of evolution.

### Dose Response Measurements

Glycerol stocks (20% glycerol) of strains containing the plasmids of interest were inoculated into 1 mL of LB media and grown overnight at 37° C. 20 μL of overnight culture was seeded into 900 μL of LB media containing ampicillin and chloramphenicol within a 2 mL 96-deep-well plate sealed with an AeraSeal film. Following growth at 37° C., 250 r.p.m. for 2 h, cultures were induced with 100 μL of a LB media solution containing appropriate antibiotics and the inducer molecule dissolved in 10 μL of DMSO. Cultures were grown for an additional 4 hours at 37° C., 250 r.p.m and subsequently centrifuged (3,500 g, 4° C., 10 min). Supernatant was removed and cell pellets were resuspended in 1 mL of PBS. 100 μL of the cell resuspension for each condition was transferred to a 96 well microtiter plate, from which the fluorescence (Ex: 485 nM, Em: 509 nM) and absorbance (600 nM) was measured using the Tecan Infinite M1000 plate reader.

### Orthogonality Assays

For each evolutionary lineage (for example, WT, THP1, THP2, THP3, THP4) all regulators were expressed on the pReg plasmid using the same promoter, which is P114-RBS (riboJ), P114-RBS (riboJ), P103-RBS (elvJ), P114-RBS (riboJ), and P103-RBS (riboJ) for the GLAU, NOS, PAP, ROTU, and THP lineages, respectively. These plasmids were co-transformed with pGFP and the following day three individual colonies were picked into LB and grown overnight. Fluorescence assays were performed as in the “Dose response measurements” section above, but either 100 mM of each BIA in 1% DMSO or DMSO itself was used for induction.

### Protein Purification

Coding sequences for RamR variants were cloned into an ampicillin resistant pUC plasmid with a T7 RNA polymerase promoter driving the gene of interest with an N-terminal His6-3C tag. Plasmids were transformed into electrocompetent BL21 DE3 cells and single transformants were grown to saturation in LB supplemented with 1,000 μg/mL carbenicillin. Cultures were diluted 1/250 in terrific broth supplemented with antibiotics in baffled flasks and incubated at 37° C. with agitation (250 r.p.m.) until reaching mid-log phase. Protein expression was induced by addition of IPTG to achieve a final concentration of 0.5 mM. For PAP4 only, papaverine was also added during IPTG induction to reach a final concentration of 100 uM. Cells were cultured for 18 hours at 18° C. Cells were harvested by centrifugation at 8,000 g for 10 min and the cell pellets were resuspended in 25 mL of wash buffer (50 mM K2HPO4, 300 mM NaCl, and 10% glycerol at pH 8.0) with protease inhibitor cocktail (complete, mini EDTA free, Roche) and lysozyme (0.5 mg/mL). Cells were incubated for 20 min at 4° C. with gentle agitation and lysed by sonication (Model 500, Fisher Scientific). Lysate was repeatedly clarified by centrifugation (35,000 g for 30 min), and protein was recovered by immobilized metal ion affinity chromatography (IMAC) using Ni-NTA resin and gravity flow columns. Eluate was concentrated and dialyzed, with 3C protease added to the dialysis cassette, into the appropriate buffer followed by purification to apparent homogeneity by size exclusion fast protein liquid chromatography (FPLC). All RamR variants were dialyzed into 20 mM Tris (pH 8.0), 200 mM NaCl and 3 mM DTT.

### X-Ray Crystallography

To form co-crystals of RamR variants in complex with individual ligands, 1 mM substrate was added to 10 mg/ml of purified protein and incubated overnight at 4° C. except for PAP4 protein, which already formed complex with papaverine during the protein expression step. Rod-shaped co-crystals grew by using sitting-drop vapor diffusion method at room temperature for PAP4, ROTU, GLAU4, and NOS4 in conditions containing 0.1M MES (pH 6.0-7.5), 14-23% PEG 3350, 0.2M Ammonium Sulfate, and 0.1M Sodium Chloride. Individual crystals were flash-frozen directly in liquid nitrogen after brief incubation with a reservoir solution supplemented with 25% (v/v) glycerol. X-ray diffraction data were collected at BL 5.0.1 beamline in ALS (Berkeley, CA). X-ray diffraction was processed to 1.6 Å, 1.8 Å, 2.0 Å, and 2.2 Å resolution for PAP4 with papaverine, ROTU4 with rotundine, GLAU4 with glaucine, and NOS4 with noscapine using HKL2000. In Phenix software, phases were obtained by molecular replacement using a previously solved RamR wildtype structure as the initial search model (PDB code 3VVX). The molecular replacement solutions for each structure were iteratively built using Coot and Phenix refine package. The quality of the final refined structures was evaluated by MolProbity. The final statistics for data collection and structure determination are shown in Table 2.

### Statistical Analysis and Reproducibility.

All data in the manuscript are displayed as mean±s.e.m. unless specifically indicated. Bar graphs, fluorescence/growth curves, dose response functions, and orthogonality matrices were all plotted in Python 3.6.9 using matplotlib and seaborn. Dose response curves and EC₅₀ values were estimated by fitting to the hill equation y=d+(a−d)*x^{b}/(c^{b}+x^{b}) (where y=output signal, b=hill coefficient, x=ligand concentration, d=background signal, a=the maximum signal, and c=the EC₅₀), with the scipy.optimize.curve_fit library in Python.

It will be apparent to those skilled in the art that various modifications and variations can be made in the present disclosure without departing from the scope or spirit of the invention. Other embodiments of the disclosure will be apparent to those skilled in the art from consideration of the specification and practice of the methods disclosed herein. It is intended that the specification and examples be considered as exemplary only, with a true scope and spirit of the invention being indicated by the claims.

TABLE 1
Key parameters of each round of RamR evolution. Each row
indicates the round of evolution (“Template → Sensor variant”),
the amount of the target BIA applied to the LB agar plate for
screening (“Alkaloid used (μM)”), the promoter/RBS used to
express the RamR variant template undergoing evolution
(“promoter expressing template”), and the libraries used to
introduce diversity (“Libraries used”). For column three, the
colored box represents the relative expression level with red
being strongest, orange being medium, and yellow being the
weakest. For column four, letter codes represent the following
(Y = yellow = T85, I88, Y92. C = cyan = K63, L66, M70.
P = purple = E121, A124, D125. B = blue = L134, C135, S138.
G = grey = R148, D152, L156. E = random mutagenesis)

| Template → | Alkaloid | Promoter | Libraries |
|---|---|---|---|
| Sensor variant | used (uM) | expressing template | used |
| WT → GLAU1 | 100 | P103-RBS(elvj) [symbol] | Y, C, G |
| GLAU1 → GLAU2 | 25 | P103-RBS(elvj) [symbol] | Y, G, E |
| GLAU2 → GLAU3 | 5 | P103-RBS(elvj) [symbol] | P, B, E |
| GLAU3 → GLAU4 | 1 | P114-RBS5 [symbol] | P, G, E |
| WT → NOS1 | 100 | P103-RBS(elvj) [symbol] | Y, C, G |
| NOS1 → NOS2 | 50 | P103-RBS(elvj) [symbol] | Y, G, E |
| NOS2 → NOS3 | 25 | P106-RBS5 [symbol] | P, Y, B, E |
| NOS3 → NOS4 | 2.5 | P114-RBS5 [symbol] | Y, B, E |
| WT → PAP1 | 200 | P103-RBS(elvj) [symbol] | Y, C, G |
| PAP1 → PAP2 | 100 | P103-RBS(elvj) [symbol] | C, G, E |
| PAP2 → PAP3 | 25 | P103-RBS(elvj) [symbol] | P, B, E |
| PAP3 → PAP4 | 2.5 | P103-RBS(elvj) [symbol] | P, G, E |
| WT → ROTU1 | 100 | P103-RBS(elvj) [symbol] | Y, C, G |
| ROTU1 → ROTU2 | 25 | P103-RBS(elvj) [symbol] | Y, E, G |
| ROTU2 → ROTU3 | 10 | P106-RBS5 [symbol] | P, Y, B, E |
| ROTU3 → ROTU4 | 2.5 | P114-RBS5 [symbol] | P, B, E |
| WT → THP1 | 50 | P103-RBS(elvj) [symbol] | Y, C, G |
| THP1 → THP2 | 5 | P103-RBS(elvj) [symbol] | C, G, E |
| THP3 → THP3 | 2 | P106-RBS5 [symbol] | P, B, E |
| THP3 → THP4 | 1 | P114-RBS5 [symbol] | P, G, E |

TABLE 2
X-ray Crystallography Data Collection and Refinement Statistics

| | | | |
|---|---|---|---|
| PAP4 | ROTU4 | NOS4 | GLAU4 |

Data collection

| | | | | |
|---|---|---|---|---|
| Space group | C2 | P1 | P1 | P1 |
| Cell dimensions | | | | |
| a, b, c (Å) | 106.76, 68.57, 69.57 | 46.14, 50.84, 50.83 | 41.63. 54.86, 92.64 | 43.18, 54.20, 91.52 |
| α, β, γ (°) | 90.00, 127.53, 90.00 | 120.05. 90.17, 89.96 | 74. [symbol] 4, 81.82, 89.96 | 104.90, 98.00, 89.99 |
| Resolution (Å) | 50.00-1. [symbol] (1.63-1.60)* | 50.00-1.73 (1.76-1.73) | 50.00-2.21 (2.25-2.21) | 50.00-2.00 (2.03-2.00) |
| Rsym/ Rplm | 0.055(0.474)/0.034(0.306) | 0.060(0.285)/0.056(0.263) | 0.072(0.354)/0.068(0.341) | 0.061(0.320)/0.056(0.300) |
| CC½^{Υ} | 0.947 (0.751) | 0.936 (0.830) | 0.921 (0.76) | 0.943 (0.813) |
| [symbol] / [symbol] | 21.3 (1.7) | 16.6 (1.9) | 12.1 (1.6) | 14.7 (1.7) |
| Completeness (%) | 99.8 (99.5) | 96.0 (95.1) | 95.0 (96.2) | 94.3 (84.2) |
| Redundancy | 3.5 (3.2) | 1.9 (1.8) | 1.8 (1.8) | 1.8 (1.5) |

Refinement

| | | | | |
|---|---|---|---|---|
| Resolution (Å) | 4 [symbol] .82-1.60 (1.66-1. [symbol] 0) | 46.14-1.74 (1.80-1.74) | 44.07-2.21 (2.29-2.21) | 43.76-2.00 (2.07-2.00) |
| No reflections | 52242 (50 [symbol] 4) | 39481 (364 [symbol] ) | 36872 (3591) | 50140 (4375) |
| R [symbol] | 0.1868 (0.2380) | 0.2162 (0.2855) | 0.2418 (0.2837) | 0.2022 (0.2552) |
| R [symbol] | 0.2108 (0.2635) | 0.2577 (0.2714) | 0.2849 (0.3413) | 0.2334 (0.2938) |
| No. atoms | 3239 | 3140 | 5950 | 6345 |
| Protein | 2935 | 2842 | 5657 | 5865 |
| Ligand/Ion | 60 | 62 | 140 | 124 |
| Water | 244 | 236 | 153 | 356 |
| B-factors (Å [symbol] ) | | | | |
| Protein | 25.4 | 23.3 | 51.7 | 42.9 |
| Ligand/Ion | 16.8 | 21.2 | 53.4 | 31.7 |
| Water | 3 [symbol] .6 | 31.7 | 48.4 | 45.1 |
| R.m.s. deviations | | | | |
| Bond lengths (Å) | 0.006 | 0.00 [symbol] | 0.01 | 0.008 |
| Bond angles (°) | 0.72 | 0.48 | 0.84 | 0.75 |
| Ramachandran plot | | | | |
| Favored | 99.45% | 99.14% | 99.15% | 99.31% |
| Allowed | 0.55% | 0.86% | 0.85% | 0.69% |
| Outliers | 0.00% | 0.00% | 0.00% | 0.00% |
| Molprobity score | 1.22/99th percentile | 1.51/99th percentile | 1.77/99th percentile | 1.53/99th percentile |

*Values for the corresponding parameters in the outermost shell in parenthesis.
^{Υ}CC_{1/2} is the Pearson correlation coefficient for a random half of the data, the two numbers represent the lowest and highest resolution shell respectively.
^{±}R_{free} is the R_{work} calculated for about 10% of the reflections randomly selected and omitted from refinement. [symbol]
indicates data missing or illegible when filed

### REFERENCES

1. (Ro, DK., Paradise, E., Ouellet, M. et al. Production of the antimalarial drug precursor artemisinic acid in engineered yeast. Nature 440, 940-943 (2006).
2. Luo, X., Reiter, M. A., d′Espaux, L. et al. Complete biosynthesis of cannabinoids and their unnatural analogues in yeast. Nature 567, 123-126 (2019).
3. Science. 2015 Sep. 4; 349 (6252): 1095-1100.
4. Nakagawa, A. et al. Total biosynthesis of opiates by stepwise fermentation using engineered Escherichia coli. Nat. Commun. 7:10390 doi: 10.1038/ncomms10390 (2016).
5. Srinivasan, P., Smolke, C. D. Biosynthesis of medicinal tropane alkaloids in yeast. Nature 585, 614-619 (2020).
6. C. Eric Hodgmanab and Michael C. Jewett, Cell-free synthetic biology: Thinking outside the cell. 2012. Metabolic engineering.
7. Metab Eng. 2021 January; 63:102-125. doi: 10.1016/j.ymben.2020.09.004. Epub 2020 Oct. 2.
8. Transcription factor-based biosensors: a molecular-guided approach for natural product engineering. Curr Opin Biotechnol. 2021. doi: 10.1016/j.copbio.2021.01.008
   8*. Genetic Biosensor Design for Natural Product Biosynthesis in Microorganisms. 2020. Trends in Biotechnology.
   8**. Hanko, E. K. R., Paiva, A. C., Jonczyk, M. et al. A genome-wide approach for identification and characterisation of metabolite-inducible systems. Nat Commun 11, 1213 (2020).
9. Della Corte, D., van Beek, H. L., Syberg, F. et al. Engineering and application of a biosensor with focused ligand specificity. Nat Commun 11, 4851 (2020)
   9* Developing a highly efficient hydroxytyrosol whole-cell catalyst by de-bottlenecking rate-limiting steps. Nature Communications.
   9** Evolution-guided engineering of small-molecule biosensors. Nucleic Acids Research.
   9*** Switching the Ligand Specificity of the Biosensor XylS from meta to para-Toluic Acid through Directed Evolution Exploiting a Dual Selection System. ACS Synthetic Biology.
10. Protein engineers turned evolutionists—the quest for the optimal starting point. Current Opinion in Biotechnology. 2019. December; 60 (12): 46-52
11. 2015. Expanding the Enzyme Universe: Accessing Non-Natural Reactions by Mechanism-Guided Directed Evolution.
12. 2012. Directed enzyme evolution: beyond the low-hanging fruit
13. 2010. MD recognition by MDR gene regulators. Herschel Wade. Current Opinion Structural Biology. Volume 20, Issue 4, August 2010, Pages 489-496
14. Improving key enzyme activity in phenylpropanoid pathway with a designed biosensor. Metabolic Engineering. Volume 40, March 2017, Pages 115-123
15. Regulatory control circuits for stabilizing long-term anabolic product formation in yeast. Metab Eng. 2020 September; 61:369-380. doi: 10.1016/j.ymben.2020.07.006. Epub Jul. 24, 2020.
16. Complete biosynthesis of noscapine and halogenated alkaloids in yeast. PNAS. 2018 Apr. 24; 115 (17). https://doi.org/10.1073/pnas.1721469115
17. Structure-Guided Engineering of a Scoulerine 9-O-Methyltransferase Enables the Biosynthesis of Tetrahydropalmatrubine and Tetrahydropalmatine in Yeast. Smolke. ACS Catalysis.
18. Genomic mining of prokaryotic repressors for orthogonal logic gates. Voigt. 2014. Nature Chemical Biology.
   18*. nalD Encodes a Second Repressor of the mexAB-oprM Multidrug Efflux Operon of Pseudomonas aeruginosa. 2006. J Bacteriology.
   18**. Cloning and characterization of SmeT, a repressor of the Stenotrophomonas maltophilia multidrug efflux pump SmeDEF. 2002. Antimicrob Agents Chemother.
19. The crystal structure of multidrug-resistance regulator RamR with multiple drugs. Nature Communications. 2013.
20. Bleomycin resistance conferred by a drug-binding protein. FEBS Letter. 1988.
21. Accelerating the semisynthesis of alkaloid-based drugs through metabolic engineering. 2017. Nature Chemical Biology.
22. 3′O-Methyltransferase, Ps3′OMT, from opium poppy: involvement in papaverine biosynthesis. 2019. Plant Cell Reports.
23. Fermentative production of tetrahydropapaverine and its derivatives using Escherichia coli. Akira NAKAGAWA.
24. Isolation and Characterization of O-methyltransferases Involved in the Biosynthesis of Glaucine in Glaucium flavum. 2015 Facchini. Plant Physiology.
25. Synthetic biology strategies for microbial biosynthesis of plant natural products. 2019. Smolke. Nature Communications.
26. Design of an in vitro biocatalytic cascade for the manufacture of islatravir. 2019. Science.
27. The nature of chemical innovation: new enzymes by evolution. 2015. Q Rev Biophys.
28. Crystal structure of the multidrug resistance regulator RamR complexed with bile acids. 2019. Sci Rep
29. Isoquinolines: Important Cores in Many Marketed and Clinical Drugs. 2021. Anticancer Agents Med Chem.
30. Privileged Scaffolds for Library Design and Drug Discovery. 2015. Curr Opin Chem Biol.
31. Dynamic control of toxic natural product biosynthesis by an artificial regulatory circuit. 2020. Metabolic Engineering
32. Synthetic addiction extends the productive life time of engineered Escherichia coli populations. PNAS. 2018.
33. An ingestible bacterial-electronic system to monitor gastrointestinal health. 2018. Science.
34. Cell-free biosensors for rapid detection of water contaminants. 2021. Nat Biotechnol.
35. Cascaded amplifying circuits enable ultrasensitive cellular sensors for toxic metals. 2019. Nat Chem Biol.
36. Harnessing the central dogma for stringent multi-level control of gene expression. 2021. Nat Comm.
37. A suppressor tRNA-mediated feedforward loop eliminates leaky gene expression in bacteria. 2021. NAR.
38. Regulation by tetracycline of gene expression in Saccharomyces cerevisiae. 1997. Molecular and General Genetics MGG.
39. Tight control of gene expression in mammalian cells by tetracycline-responsive promoters. 1992.
40. Stringent repression and homogeneous de-repression by tetracycline of a modified CaMV 35S promoter in intact transgenic tobacco plants. 1992. Plant J.

We claim:

1. A biosensor comprising an engineered substrate-promiscuous regulator, wherein said substrate-promiscuous regulator has been engineered to interact more efficiently with an input signal than does the naturally occurring substrate promiscuous regulator; and further wherein the biosensor is engineered to provide an output signal, wherein said output signal is generated in response to interaction with the input signal.

2. The biosensor of claim 1, wherein the naturally occurring regulator from which the engineered biosensor is derived is RamR of Salmonella typhimurium.

3. The biosensor of claim 1, wherein the engineered biosensor has about 97% to 99% identity to QacR (WP_001807342.1), TtgR (WP_010952495.1), SmeT (WP_005414519.1), NalD (WP_003092152.1), LmrR (WP_011834386.1), EbrR (WP_003976902), MexR (WP_003114897.1), LadR (WP_003721913.1), VceR (WP_001264144.1), MttR (WP_003693763.1), AcrR (WP_000101737), MepR (WP_000397416.1), SCO4008 (WP_011029378.1), Rv3066 (WP_003416005.1), CgmR (WP_011015249.1), CmeR (WP_002857627.1), Rv0302 (WP_003401571.1), BepR (WP_004687968.1), MexL (WP_003092468.1), TtgT (WP_012052586.1), TtgV (WP_014003968.1), LmrA (WP_003246449.1), TM_1030 (WP_010865247.1) or Bm3R1 (WP_013083972.1), or RamR (WP_000113609.1)

4. The biosensor of any one of claims 1-3, wherein said input signal is a naturally occurring composition.

5. The biosensor of any one of claims 1-3, wherein said input signal is a synthetic composition and is not naturally occurring.

6. The biosensor of claim 4, wherein the naturally occurring composition is a plant alkaloid.

7. The biosensor of claim 6, wherein said plant alkaloid is tetrahydropapaverine, papaverine, rotundine, glaucine, noscapine, norbelladine, or 4-o-methylnorbelladine

8. The biosensor of any one of claims 1-7, wherein the output signal is expression of a gene.

9. The biosensor of any one of claims 1-8, wherein the output signal is fluorescence, luminescence, or a colorimetric signal.

10. The biosensor of any one of claims 1-9, wherein the input signal is converted to the output signal by a transduction system.

11. The biosensor of claim 10, wherein the transduction system comprises a transcriptional activator or transcriptional repressor of the output signal.

12. The biosensor of claim 11, wherein the transcriptional activator or transcriptional repressor is encoded with the engineered substrate promiscuous regulator.

13. The biosensor of claim 11 or 12, wherein the transduction system comprises a promoter or operator and a regulator.

14. The biosensor or any one of claims 1-13, wherein the biosensor is 90% or more identical to the naturally occurring form of the substrate promiscuous regulator.

15. The biosensor of any one of claims 1-14, wherein said interaction with the input signal occurs via a covalent or a non-covalent bond.

16. The biosensor of any one of claims 1-15, wherein the substrate promiscuous regulator comprises a large hydrophobic binding pocket.

17. The biosensor of any one of claims 1-16, wherein the substrate promiscuous regulator is a multidrug resistance regulator.

18. A plasmid comprising a nucleic acid encoding the biosensor of any one of claims 1-17.

19. The plasmid of claim 19, wherein said plasmid further comprises a nucleic acid encoding the output signal.

20. A cell comprising the plasmid of claim 18 or 19

21. The biosensor of any one of claims 1-17, wherein the biosensor is integrated into a host genome of a cell.

22. The cell of claim 20 or 21, wherein the cell is further engineered to produce a product of interest.

23. The cell of any one of claims 20-22, wherein said cell is a eukaryote or a prokaryote.

24. A method of making a product of interest, the method comprising
a. providing the recombinant host cell of claim 22 or 23; and
b. contacting the recombinant host cell with reagents needed to produce the product under conditions whereby a product is produced.

25. A method of engineering a substrate-promiscuous regulator to function as a biosensor, the method comprising:
a. identifying a naturally occurring substrate-promiscuous regulator;
b. engineering the naturally occurring substrate-promiscuous regulator for increased sensitivity to an input signal when compared to the naturally occurring substrate promiscuous regulator;
c. introducing into a cell:
i. nucleic acid encoding the engineered substrate-promiscuous regulator of step b), and
ii. a transduction system for providing an output signal, wherein said output signal is generated in response to interaction with the input signal;
d. exposing the cell of step c) to the input signal; and
e. detecting an output signal; wherein detection of said output signal indicates a functional biosensor.

26. The method of claim 25, wherein said substrate-promiscuous regulator is naturally occurring in a prokaryotic organism.

27. The method of claim 25 or 26, wherein in step b), said engineering occurs via genetic mutation of the naturally occurring substrate-promiscuous regulator.

28. The method of claim 27, wherein said engineering comprises chip-based DNA synthesis, CRISPR, multiplexed genome engineering, in vivo mutagenesis, random mutagenesis, recombineering, or site-directed mutagenesis.

29. The method of claim 27, wherein said engineering comprises determining a hotspot for potential input signal recognition and creating mutations within the hotspot to create an engineered substrate-promiscuous regulator.

30. The method of any one of claims 25-29, wherein said input signal is converted to the output signal by a transduction system.

31. The method of claim 30, wherein the transduction system comprises a transcriptional activator or transcriptional repressor of the output signal.

32. The method of claim 31, wherein the transcriptional activator or transcriptional repressor is encoded by the engineered substrate-promiscuous regulator.

33. The method of claim 31, wherein the transduction system comprises a promoter or operator and a regulator.

34. The method of any of claims 25-33, wherein said input signal is a naturally occurring composition.

35. The method of any of claims 25-34, wherein said input signal is a synthetic composition and is not naturally occurring.

36. The method of any one of claims 25-35, wherein the cell is a prokaryotic or eukaryotic cell.

37. A kit comprising a biosensor comprising an engineered substrate-promiscuous regulator, wherein said substrate-promiscuous regulator has been engineered to interact more efficiently with an input signal than does the naturally occurring substrate promiscuous regulator.

38. The kit of claim 37, further comprising an output signal; wherein said output signal is generated in response to interaction with the input signal.

39. The kit of claim 37 or 38, wherein the biosensor and the output signal are encoded in a plasmid.

40. The kit of claim 39, wherein the kit further comprises components required for transformation of the plasmid into a cell.

41. The kit of claim 39, wherein the kit comprises a cell transformed with the plasmid.

42. The kit of claim 37, wherein the biosensor and the output signal of the kit are engineered so that they can be integrated in a genome of a cell.

43. The kit of claim 40, wherein the kit comprises a cell integrated with the biosensor and the output signal.

44. The kit of any one of claims 37-43, wherein the kit further comprises components needed for detection of expression of a target molecule.

45. The kit of any one of claims 37-44, wherein the output signal is expression of a gene.

46. The kit of any one of claims 37-44, wherein the output signal is fluorescence, luminescence, or a colorimetric signal.

47. The kit of any one of claims 37-46, wherein the kit further comprises a transduction system, wherein the transduction system converts the input signal to the output signal.

48. The kit of claim 47, wherein the transduction system comprises a transcriptional activator or transcriptional repressor of the output signal, wherein the transduction system is encoded with the engineered substrate promiscuous regulator.

49. The kit of claim 48, wherein the transduction system comprises a promoter or operator and a regulator.

50. A nucleic acid comprising 97% or more identity to any one of SEQ ID NOS: 1-6.


SEQUENCE LISTING (18 sequences)

SEQ ID NO: 1 - protein, 194 aa, Artificial sequence
  Description of sequence peptide sensor
  Name GLAU4
  This sensor binds to glaucine
MVARPKSEDKKQALLEAATQAIAQSGIAASTAVIARNAGVAEGTLFRYFATKDELINTLYLHLTQDWCQSIIMELDRSITDAKMMTRFLWNSWISWGLNHPARHRAIRQLAVSEKLTKETEQRADDMFPELRDHLHRNVLMVFMSDEYRAFGDGLFLALAETTMDFAARDPARAGEYIALGFEAMWRALAREEQ

SEQ ID NO: 2 - protein, 194 aa, Artificial sequence
  Description of sequence peptide sensor
  Name NOS4
  This sensor binds to noscapine
MVARPKSEDKKQALLEAATQAIAQSGIAASTAVIARNAGVAEGTLFRYFATKDELINTLYLHLTHDMCQSLIMELDRSITDAKMMTRFIWNSYISWGLNHPARHRAIRQLAVSEKLTKETRQRARDMFPELRDLCYRSLLMVFMSDEYRAFGDGLFMALAETTMDFAARDPARAGEYIALGFEAMWRALTREEQ

SEQ ID NO: 3 - protein, 194 aa, Artificial sequence
  Description of sequence peptide sensor
  Name PAP4
  This sensor binds to papaverine
MVARPKSEDKKQALLEAATQAIAQSGIAASTAVIARNAGVAEGTLFRYFATKDELINTLYLHLRQDLCQSLIMELDRSITDAKMMMRFIWNSGISWGLNHPARHRAIRQLAVSEKLTKETHQRDLDMFPELRDILHRRVLMVFMSDEYRAFGDGLFLALAETTMDFAARDPARAGEYIALGFEAMWRALTREEQ

SEQ ID NO: 4 - protein, 194 aa, Artificial sequence
  Description of sequence peptide sensor
  Name ROTU4
  This sensor binds to rotundine
MVARPKSEDKKQALLEAATQAIAQSGIAASTAVIARNAGVAEGTLFRYFATKDELINTLYLHLYQDHCQSLIMELDRSITDAKMMIRFTWNSYISWGLNHPARHRAIRQLAVSEKLTKETKQRIEDMFPELRDILHRSVLMVFMSDEYSAFGKGLFYALAETTMDFAARDPARAGEYIALGFEAMWRALTREEQ

SEQ ID NO: 5 - protein, 194 aa, Artificial sequence
  Description of sequence peptide sensor
  Name THP4
  This sensor binds to tetrahydropapaverine
MVARPKSEDKKQALLEAATQAIAQSGTAASTAVIARNAGVAEGTLFRYFATKDELINTLYLHLFQDWCQSSIMELDRSITDAKMMTRFLWNSIISWGLNHPARHRAIRQLAVSEKLSKETVQRADDMFPELRDIVHREVLMVFMSDEYRAFGEGLFLALAETTMDFAARDPARAGEYIALGFEAMWRALTREEQ

SEQ ID NO: 6 - protein, 194 aa, Artificial sequence
  Description of sequence peptide sensor
  Name 4NB2
  This sensor binds to 4-Omethylnorbelladine
MVARPKSEDKKQALLEAATQAIAQSGIAASTAVIARNAGVAEGTLFRYFATKDELINTLYLHLTQDMCQSMIMELDRSITDAKMMTRFIWNSYISWGLNHPARHRAIRQLAVSEKLTKETEQRADDMFPELRDLDHRGVLMVFMSDEYRAFGDGLFLALAETTMDFAARDPARAGEYIALGFEAMWRALTREEQ

SEQ ID NO: 7 - DNA, 585 bp, Artificial sequence
  Description of sequence encodes for peptide sensor
  DNA encoding for GLAU4
atggttgctcgcccaaagtctgaggacaaaaagcaggcattgcttgaagcggcaactcaagccatcgcgcaatcaggcattgccgctagtaccgctgtaattgcacgcaatgcgggagttgcggaagggacgttgttccgctatttcgcaacgaaagatgagttgatcaacaccctttacttacatttgacccaggactggtgccaatcaatcatcatggaattggatcgttctattactgacgctaagatgatgacccgttttttgtggaacagttggattagctggggattgaaccacccagctcgccatcgtgccattcgtcagttggcggtttctgaaaagttgacgaaggaaaccgaacaacgcgcggatgatatgttcccggagttacgcgaccacctgcaccgtaacgttcttatggtgtttatgtccgacgagtaccgcgccttcggcgacgggttgttcttggcgcttgctgagacgactatggatttcgctgcgcgcgacccggctcgcgctggtgagtacattgcgttgggcttcgaggctatgtggcgcgcattggcccgcgaagagcagtaa

SEQ ID NO: 8 - DNA, 585 bp, Artificial sequence
  Description of sequence encodes for peptide sensor
  DNA encoding for NOS4
atggttgctcgcccaaagtctgaggacaaaaagcaggcattgcttgaagcggcaactcaagccatcgcgcaatcaggcattgccgctagtaccgctgtaattgcacgcaatgcgggagttgcggaagggacgttgttccgctatttcgcaacgaaagatgagttgatcaacaccctttacttacatttgacccatgacatgtgccaatcactgatcatggaattggatcgttctattactgacgctaagatgatgacccgttttatctggaacagttatattagctggggattgaaccacccagctcgccatcgtgccattcgtcagttggcggtttctgaaaagttgacgaaggaaacccgccaacgcgcccgcgatatgttcccggagttacgcgacttgtgctaccgtagtttgcttatggtgtttatgtccgacgagtaccgcgccttcggcgacgggttgttcatggcgcttgctgagacgactatggatttcgctgcgcgcgacccggctcgcgctggtgagtacattgcgttgggcttcgaagctatgtggcgcgcacttacgcgcgaagagcagtaa

SEQ ID NO: 9 - DNA, 585 bp, Artificial sequence
  Description of sequence encodes for peptide sensor
  DNA encoding for PAP4
atggttgctcgcccaaagtctgaggacaaaaagcaggcattgcttgaagcggcaactcaagccatcgcgcaatcaggcattgccgctagtaccgctgtaattgcacgcaatgcgggagttgcggaagggacgttgttccgctatttcgcaacgaaagatgagttgatcaacaccctttacttacatttgaggcaggacctgtgccaatcactcatcatggaattggatcgttctattactgacgctaagatgatgatgcgttttatctggaacagtggcattagctggggattgaaccacccagctcgccatcgtgccattcgtcagttggcggtttctgaaaagttgacgaaggaaacccaccaacgcgacctggatatgttcccggagttacgcgacatcctgcaccgtagggttcttatggtgtttatgtccgacgagtaccgcgccttcggcgacgggttgttcttggcgcttgctgagacgactatggatttcgctgcgcgcgacccggctcgcgctggtgagtacattgcgttgggcttcgaggctatgtggcgcgcacttacgcgcgaagagcagtaa

SEQ ID NO: 10 - DNA, 585 bp, Artificial sequence
  Description of sequence encodes for peptide sensor
  DNA encoding for ROTU4
atggttgctcgcccaaagtctgaggacaaaaagcaggcattgcttgaagcggcaactcaagccatcgcgcaatcaggcattgccgctagtaccgctgtaattgcacgcaatgcgggagttgcggaagggacgttgttccgctatttcgcaacgaaagatgagttgatcaacaccctttacttacatttgtaccaggaccactgccaatcactgatcatggaattggatcgttctattactgacgctaagatgatgatccgttttacctggaacagttacattagctggggattgaaccacccagctcgccatcgtgccattcgtcagttggcggtttctgaaaagttgacgaaggaaaccaagcaacgcatcgaggatatgttcccggagttacgcgacatcctgcaccgtagtgttcttatggtgtttatgtccgacgagtactccgccttcggcaaggggttgttctacgcgcttgctgagacgactatggatttcgctgcgcgcgacccggctcgcgctggtgagtacattgcgttgggcttcgaggctatgtggcgcgcacttacgcgcgaagagcagtaa

SEQ ID NO: 11 - DNA, 585 bp, Artificial sequence
  Description of sequence encodes for peptide sensor
  DNA encoding for THP4
atggttgctcgcccaaagtctgaggacaaaaagcaggcattgcttgaagcagcaactcaagccatcgcgcaatcaggcactgccgctagtaccgctgtaattgcacgcaatgcgggagttgcggaagggacgttgttccgctatttcgcaacgaaagatgagttgatcaacaccctttacttacatttgttccaggactggtgccaatcatccatcatggaattggatcgttctattactgacgctaagatgatgacgcgttttctctggaacagtatcattagctggggattaaaccacccagctcgccatcgtgccattcgtcagttggcggtttctgaaaagttgtcgaaggagaccgtacaacgcgcggatgatatgttcccggagttacgcgacatcgtccaccgtgaggttcttatggtgtttatgtccgacgagtaccgcgccttcggcgaagggttgttcttggcgcttgctgagacgactatggatttcgctgcgcgcgacccggctcgcgctggtgagtacattgcgttgggcttcgaggctatgtggcgcgcacttacgcgcgaagagcagtaa

SEQ ID NO: 12 - DNA, 585 bp, Artificial sequence
  Description of sequence encodes for peptide sensor
  DNA encoding for 4NB2
atggttgctcgcccaaagtctgaggacaaaaagcaggcattgcttgaagcggcaactcaagccatcgcgcaatcaggcattgccgctagtaccgctgtaattgcacgcaatgcgggagttgcggaagggacgttgttccgctatttcgcaacgaaagatgagttgatcaacaccctttacttacatttgacccaggacatgtgccaatcaatgatcatggaattggatcgttctattactgacgctaagatgatgacccgttttatctggaacagttatattagctggggattgaaccacccagctcgccatcgtgccattcgtcagttggcggtttctgaaaagttgacgaaggaaaccgaacaacgcgcggatgatatgttcccggagttacgcgacctcgaccaccgtggcgttcttatggtgtttatgtccgacgagtaccgcgccttcggcgacgggttgttcttggcgcttgctgagacgactatggatttcgctgcgcgcgacccggctcgcgctggtgagtacattgcgttgggcttcgaggctatgtggcgcgcacttacgcgcgaagagcagtaa

SEQ ID NO: 13 - DNA, 77 bp, Artificial sequence
  Description of sequence GLAU variant
  Name Pbm3r1
ggctgtcggacctttgaaaagttcgttgacagctagctcagtcctaggtacaatcggaatgaacgttcattccgttt

SEQ ID NO: 14 - DNA, 61 bp, Artificial sequence
  Description of sequence GLAU variant
  Name Pramr
ctttgaaaagtaccttgacggcgtatctttgctttctataatgagtgcttactcactcata

SEQ ID NO: 15 - DNA, 69 bp, Artificial sequence
  Description of sequence GLAU variant
  Name Pqacr
attcgttaccaattgacagctagctcagtcctactttagtatatagagactgagcggtcggtctatata

SEQ ID NO: 16 - DNA, 56 bp, Artificial sequence
  Description of sequence GLAU variant
  Name Pttgr
aacgcacccagcagtatttacaaacaaccatgaatgtaagtataatagttagcaat

SEQ ID NO: 17 - DNA, 52 bp, Artificial sequence
  Description of sequence GLAU variant
  Name Pnald
cacctttcggacgtttacaaacacctatgaatgtaagtataatagttagcaa

SEQ ID NO: 18 - DNA, 52 bp, Artificial sequence
  Description of sequence GLAU variant
  Name Psmet
cacccagcagtatttacaaacaaacaagcatgtatgtataatccttagcaac